SEQUENCE LISTING (27 sequences)

SEQ ID NO: 1 - DNA, 1609 bp, Rattus norvegicus
gtgtgctggagccactgtcgccgatctcgcgcacgctactgctgctgctcgcccgtcgtcccccatcgtgcactaagcggtcccaaaagattcaaagtccaagatggcagccctcaaggaccagctgattgtgaatcttcttaaggaagaacaggtcccccagaacaagattacagttgttggggttggtgctgttggcatggcttgtgccatcagtatcttaatgaaggacttggctgatgagcttgcccttgttgatgtcatagaagataagctaaagggagagatgatggatcttcagcatggcagccttttccttaagacaccaaaaattgtctccagcaaagattatagtgtgactgcaaactccaagctggtcattatcaccgcgggggcccgtcagcaagagggagagagccggctcaatttggtccagcgaaacgtgaacatcttcaagttcatcattccaaatgttgtgaaatacagtccacagtgcaaactgctcatcgtctcaaacccagtggatatcttgacctacgtggcttggaagatcagcggcttccccaaaaacagagttattggaagtggttgcaatctggattcggctcggttccgttacctgatgggagaaaggctgggagttcatccactgagctgtcacgggtgggtcctgggagagcatggcgactccagtgtgcctgtgtggagtggtgtgaacgtcgccggcgtctccctgaagtctctgaacccgcagctgggcacggatgcagacaaggagcagtggaaggatgtgcacaagcaggtggttgacagtgcatacgaagtgatcaagctgaaaggttacacatcctgggccattggcctctccgtggcagacttggccgagagcataatgaagaaccttaggcgggtgcatcccatttccaccatgattaagggtctctatggaatcaaggaggatgtcttcctcagcgtcccatgtatcctgggacaaaatggaatctcagatgttgtgaaggtgacactgactcctgacgaggaggcccgcctgaagaagagtgcagataccctctggggaatccagaaggagctgcagttctaaagtcttcccagtgtcctagcacttcactgtccaggctgcagcagggtttctatggagaccacgcacttctcatctgagctgtggttagtccagttggtccagttgtgttgaggtggtctgggggaaatctcagttccacagctctaccctgctaagtggtacttgtgtagtggtaacctggttagtgtgacaatcccactgtctccaagacacactgccaactgcatgcaggctttgattaccctgtgagcctgctgcattgctgtgctacgcaccctcaccaaacatgcctaggccatgagttcccagttagttataagctggctccagtgtgtaagtccatcgtgtatatcttgtgcataaatgttctacaggatattttctgtattatatgtgtctgtagtgtacattgcaatattacgtgaaatgtaagatctgcatatggatgatggaaccaaccactcaagtgtcatgccaaggaaaacaccaaataaaccttgaacagtg

SEQ ID NO: 2 - protein, 332 aa, Rattus norvegicus
MAALKDQLIVNLLKEEQVPQNKITVVGVGAVGMACAISILMKDLADELALVDVIEDKLKGEMMDLQHGSLFLKTPKIVSSKDYSVTANSKLVIITAGARQQEGESRLNLVQRNVNIFKFIIPNVVKYSPQCKLLIVSNPVDILTYVAWKISGFPKNRVIGSGCNLDSARFRYLMGERLGVHPLSCHGWVLGEHGDSSVPVWSGVNVAGVSLKSLNPQLGTDADKEQWKDVHKQVVDSAYEVIKLKGYTSWAIGLSVADLAESIMKNLRRVHPISTMIKGLYGIKEDVFLSVPCILGQNGISDVVKVTLTPDEEARLKKSADTLWGIQKELQF

SEQ ID NO: 3 - DNA, 1287 bp, Rattus norvegicus
cgctctacttgctgtaggactctgggtgatgggagaagagcgggagggcagttctttaaccgtgtaagaggagggaccatcccttttggggttcatcaagatgagtaagaactcaggcggctacacatatacggagacctcagtattatttttccatttcaaggtcccaaaagattcaaagtccaagatggcagccctcaaggaccagctgattgtgaatcttcttaaggaagaacaggtcccccagaacaagattacagttgttggggttggtgctgttggcatggcttgtgccatcagtatcttaatgaaggacttggctgatgagcttgcccttgttgatgtcatagaagataagctaaagggagagatgatggatcttcagcatggcagccttttccttaagacaccaaaaattgtctccagcaaagattatagtgtgactgcaaactccaagctggtcattatcaccgcgggggcccgtcagcaagagggagagagccggctcaatttggtccagcgaaacgtgaacatcttcaagttcatcattccaaatgttgtgaaatacagtccacagtgcaaactgctcatcgtctcaaacccagtggatatcttgacctacgtggcttggaagatcagcggcttccccaaaaacaaagttattggaagtggttgcaatctggattcggctcggttccgttacctgatgggagaaaggctgggagttcatccactgagctgtcacgggtgggtcctgggagagcatggcgactccagtgtgcctgtgtggagtggtgtgaacgtcgccggcgtctccctgaagtctctgaacccgcagctgggcacggatgcagacaaggagcagtggaaggatgtgcacaagcaggtggttgacagtgcatacgaagtgatcaagctgaaaggttacacatcctgggccattggcctctccgtggcagacttggccgagagcataatgaagaaccttaggcgggtgcatcccatttccaccatgattaagggtctctatgggatcaaggaggatgtcttcctcagcgtcccatgtatcctgggacaaaatggaatctcagatgttgtgaaggtgacactgactcctgacgaggaggcccgcctgaagaagagtgcagataccctctggggaatccagaaggagctgcagttctaaagtcttcccagtgtcctagcacttcactgtccaggctgcagcagggtttctatggagaccacgcacttctcatctgagctgtggttagtccagttggtcca

SEQ ID NO: 4 - protein, 361 aa, Rattus norvegicus
MSKNSGGYTYTETSVLFFHFKVPKDSKSKMAALKDQLIVNLLKEEQVPQNKITVVGVGAVGMACAISILMKDLADELALVDVIEDKLKGEMMDLQHGSLFLKTPKIVSSKDYSVTANSKLVIITAGARQQEGESRLNLVQRNVNIFKFIIPNVVKYSPQCKLLIVSNPVDILTYVAWKISGFPKNRVIGSGCNLDSARFRYLMGERLGVHPLSCHGWVLGEHGDSSVPVWSGVNVAGVSLKSLNPQLGTDADKEQWKDVHKQVVDSAYEVIKLKGYTSWAIGLSVADLAESIMKNLRRVHPISTMIKGLYGIKEDVFLSVPCILGQNGISDVVKVTLTPDEEARLKKSADTLWGIQKELQF

SEQ ID NO: 5 - protein, 15 aa, Artificial Sequence
  Mitochondrial targeting peptide
MLSRLSLRLLSRYLL

SEQ ID NO: 6 - protein, 34 aa, Zea mays
MYRAAASLASKARQAGSSSAARQVGSRLAWSRNY

SEQ ID NO: 7 - protein, 24 aa, Artificial Sequence
  Mitochondrial targeting peptide
MLSLRQSIRFFPATRTLCSSRYLL

SEQ ID NO: 8 - protein, 27 aa, Artificial Sequence
  Mitochondrial targeting peptide
MLRTSSLFTRRVQPSLFSRNILRLQST

SEQ ID NO: 9 - protein, 17 aa, Artificial Sequence
  Mitochondrial targeting peptide
MLSLRQSIRFFKPATRT

SEQ ID NO: 10 - protein, 21 aa, Artificial Sequence
  Mitochondrial targeting peptide
MFSNLSKRWAQRTLSKSFYST

SEQ ID NO: 11 - protein, 10 aa, Artificial Sequence
  Mitochondrial targeting peptide
MKSFITRNKT

SEQ ID NO: 12 - DNA, 25 bp, Artificial Sequence
  Synthetic oligonucleotide primer
ggaattcgtgtgctggagccactgt

SEQ ID NO: 13 - DNA, 28 bp, Artificial Sequence
  Synthetic oligonucleotide primer
cgcggatcctgtagaacattttatgcac

SEQ ID NO: 14 - DNA, 18 bp, Artificial Sequence
  Synthetic oligonucleotide primer
cgctctacttgctgtagg

SEQ ID NO: 15 - DNA, 22 bp, Artificial Sequence
  Synthetic oligonucleotide primer
gcctggacagtgaagtgctagg

SEQ ID NO: 16 - DNA, 24 bp, Artificial Sequence
  Synthetic oligonucleotide primer
aaccgtgtaagaggagggaccatc

SEQ ID NO: 17 - DNA, 24 bp, Artificial Sequence
  Synthetic oligonucleotide primer
tggaccaactggactaaccacagc

SEQ ID NO: 18 - DNA, 174 bp, Mus musculus
ggagcaacttggcgctctacttgctgtagggctctgggtgatgggagaagagcgggagggcagctttctaaccatataagaggagataccatccccttttggttcatcaagatgagtaagtcctcaggcggctacacgtacacggagacctcggtattatttttccatttcaag

SEQ ID NO: 19 - DNA, 78 bp, Mus musculus
tgcgtgctggagccactgtcgccgagctcggccacgctgcttctcctcgccagtcgcccccccatcgtgcatctagcg

SEQ ID NO: 20 - protein, 29 aa, Mus musculus
MSKSSGGYTYTETSVLFFHFKVSKDSKSK

SEQ ID NO: 21 - protein, 29 aa, Rattus norvegicus
MSKNSGGYTYTETSVLFFHFKVPKDSKSK

SEQ ID NO: 22 - protein, 29 aa, Homo sapiens
MGEPSGGYTYTQTSIFLFHAKIPFGSKSN

SEQ ID NO: 23 - protein, 29 aa, Artificial Sequence
  Consensus leader sequence
MSKXSGGYTYTETSVLFFHFKVPKDSKSK

SEQ ID NO: 24 - DNA, 1680 bp, Mus musculus
gagcaacttggcgctctacttgctgtagggctctgggtgatgggagaagagcgggagggcagctttctaaccatataagaggagataccatccccttttggttcatcaagatgagtaagtcctcaggcggctacacgtacacggagacctcggtattatttttccatttcaaggtctcaaaagattcaaagtccaagatggcaaccctcaaggaccagctgattgtgaatcttcttaaggaagagcaggctccccagaacaagattacagttgttggggttggtgctgttggcatggcttgtgccatcagtatcttaatgaaggacttggcggatgagcttgcccttgttgacgtcatggaagacaaactcaagggcgagatgatggatctccagcatggcagcctcttccttaaaacaccaaaaattgtctccagcaaagactactgtgtaactgcgaactccaagctggtcattatcaccgcgggggcccgtcagcaagagggggagagccggctcaacctggtccagcgaaacgtgaacatcttcaagttcatcattcccaacattgtcaagtacagtccacactgcaagctgctgatcgtctccaatccagtggatatcttgacctacgtggcttggaaaatcagtggctttcccaaaaaccgagtaattggaagtggttgcaatctggattcagcgcggttccgttacctgatgggagagaggctgggggttcacgcgctgagctgtcacggctgggtcctgggagaacatggcgactccagtgtgcctgtgtggagtggtgtgaatgttgccggcgtctccctgaagtctcttaacccagaactgggcactgacgcagacaaggagcagtggaaggaggttcacaagcaggtggtggacagtgcctacgaggtgatcaagctgaaaggttacacatcctgggccattggcctctctgtggcagacttggctgagagcataatgaagaaccttaggcgggtgcatcccatttccaccatgattaagggtctctatggaatcaatgaggatgtcttcctcagtgtcccatgtatcctgggacaaaatggaatctcggatgttgtgaaggtgacactgactcctgaggaagaggcccgcctgaagaagagcgcagacaccctctggggaatccagaaggagctgcagttctaaagtcttccccgtgtcctagcacttcactgtccaggctgcagcagggcttctaggcagaccacacccttctcgtctgagctgtggttagtacagtggtgttgagatggtgtggggaaacatctcactccccacagctctgccctgctgccaagtggtacttgtgtagtggtgacctggttagtgtgacagtcccactgtctctgagacacactgccaactgcaggcttcgattacccctgtgagcctgctgcattgctgccctgcaccaaacagcctaggccgacgagttcccagttaagtcgtataacctggctccagtgtgtacgtccatgatgcatatcttgtgcataaatgttgtacaggatattttatatattatatgtgtctgtagtgtgcattgcaatattatgtgagatgtaagatctgcatatggatgatggaaccaaccacccaagtgtcatgccaaataaaaccttgaacagtg

SEQ ID NO: 25 - protein, 361 aa, Mus musculus
MSKSSGGYTYTETSVLFFHFKVSKDSKSKMATLKDQLIVNLLKEEQAPQNKITVVGVGAVGMACAISILMKDLADELALVDVMEDKLKGEMMDLQHGSLFLKTPKIVSSKDYCVTANSKLVIITAGARQQEGESRLNLVQRNVNIFKFIIPNIVKYSPHCKLLIVSNPVDILTYVAWKISGFPKNRVIGSGCNLDSARFRYLMGERLGVHALSCHGWVLGEHGDSSVPVWSGVNVAGVSLKSLNPELGTDADKEQWKEVHKQVVDSAYEVIKLKGYTSWAIGLSVADLAESIMKNLRRVHPISTMIKGLYGINEDVFLSVPCILGQNGISDVVKVTLTPEEEARLKKSADTLWGIQKELQF

SEQ ID NO: 26 - DNA, 1761 bp, Homo sapiens
ctctggtgtttacttgagaagccctggctgtgtccttgctgtaggagccggagtagctcagagtgatcttgtctgaggaaaggccagccccacttggttaataaaccgcgatgggtgaaccctcaggaggctatacttacacccaaacgtcgatattccttttccacgctaagattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtg

SEQ ID NO: 27 - protein, 361 aa, Homo sapiens
MGEPSGGYTYTQTSIFLFHAKIPFGSKSNMATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

(19) United States
(12) Patent Application Publication
(10) Pub. No.: US 2005/0014692 A1
(43) Pub. Date: Jan. 20, 2005

(54) Lactate dehydrogenase as a novel target and reagent for diabetes therapy

(76) Inventors: Christopher B. Newgard, Chapel Hill, NC (US); Mette V. Jensen, Durham, NC (US); A. Dean Sherry, Dallas, TX (US); Shawn C. Burgess, Little Elm, TX (US)

Correspondence Address: Karen A. Magri;Myers Bigel Sibley & Sajovec, P.A., P.O. Box 37428, Raleigh, NC, 27627, US

(21) Appl. No.: 10/760,644

(22) Filed: Jan. 20, 2004

Related U.S. Application Data
(60) Provisional application No. 60/441,476, filed on Jan. 21, 2003.

(51) Int. Cl. A61K 48/00; A61K 39/395; A61K 38/22

(52) U.S. Cl. 514/12; 514/44; 424/145.1

(57) ABSTRACT

The present invention provides an isolated nucleic acid encoding a novel lactate dehydrogenase (LDH) as well as the isolated LDH polypeptide. Also provided are methods of enhancing fuel-stimulated insulin secretion, in particular, glucose-stimulated insulin secretion. Further provided are methods of screening for compounds that bind LDH, modulate LDH activity, and/or modulate fuel- or glucose-stimulated insulin secretion and the compounds identified thereby. The invention further provides a method of enhancing insulin secretion by administering a nucleic acid encoding LDH to a subject in a therapeutically effective amount.

### RELATED APPLICATION INFORMATION

This application claims the benefit of U.S. Provisional Application No. 60/441,476, filed Jan. 21, 2003, which is incorporated by reference herein in its entirety.

### GOVERNMENT SUPPORT

The present invention was made, in part, with the support of grant numbers DK 42682 and RR-02584 from the National Institutes of Health. The United States government has certain rights to this invention.

### FIELD OF THE INVENTION

The present invention relates to novel targets and reagents for diabetes therapy; in particular, the use of lactate dehydrogenase as a target for drug discovery or as a therapeutic reagent.

### BACKGROUND OF THE INVENTION

Glucose induced insulin secretion (IS) is tightly coupled to the metabolism of glucose. The far dominant pathway for glucose-derived carbons in pancreatic β-cells is glycolysis. The glycolytic endpoint, pyruvate, is converted to lactate in the cytosol, a process catalyzed by lactate dehydrogenase (LDH); FIG. 1. Only the A form of LDH is believed to be expressed in islets and the activity of the enzyme is reported to be very low (Sekine et al., (1994) J. Biol. Chem. 269:4895). Thus, the vast majority of glucose-derived pyruvate will enter the mitochondria and either be converted into acetyl-CoA for subsequent oxidation in the TCA cycle, or will be carboxylated to oxaloacetate by pyruvate carboxylase. In β-cells, the amount of glucose-derived pyruvate that enters mitochondrial metabolism by carboxylation almost equals that which enters by decarboxylation (MacDonald, (1993) Arch. Biochem. Biophys. 305:205; Khan et al., (1996) J. Biol. Chem. 241:2539). It has been proposed that pyruvate carboxylase-catalyzed anaplerotic influx of pyruvate is linked to an export of NADPH equivalents from the mitochondria which in turn might be important for IS regulation (Macdonald, (1995) J. Biol. Chem. 270:20051). Furthermore, by ¹³C NMR isotopomer analyses, evidence has previously been obtained for a direct correlation between pyruvate cycling (substrate flux through pyruvate carboxylase and back to pyruvate) and IS in the INS-1 derived cell line 832/13 (Lu et al., (2002) Proc. Natl. Acad. Sci. USA 99:2708).

The participation of the pancreatic islets of Langerhans in fuel homeostasis is mediated in large part by their ability to respond to changes in circulating levels of key metabolic fuels by secreting peptide hormones. Insulin secretion from islet β-cells is stimulated by amino acids, three-carbon sugars such as glyceraldehyde, and most prominently, by glucose. The capacity of normal islet β-cells to “sense” a rise in blood glucose concentration, and to respond to elevated levels of glucose (as occurs following ingestion of a carbohydrate-containing meal) by secreting insulin is critical to control of blood glucose levels. Increased insulin secretion in response to a glucose load prevents chronic hyperglycemia in normal individuals by stimulating glucose uptake into peripheral tissues, particularly muscle and adipose tissue.

Mature insulin consists of two polypeptide chains, A and B, joined in a specific manner. However, the initial protein product of the insulin gene in β-cells is not insulin, but preproinsulin. This precursor differs from mature insulin in two ways. Firstly, it has a so-called N-terminal “signal” or “pre” sequence which directs the polypeptide to the rough endoplasmic reticulum, where it is proteolytically processed. The product, proinsulin, still contains an additional connecting peptide between the A and B chains, known as the C-peptide, which permits correct folding of the whole molecule. Proinsulin is then transported to the Golgi apparatus, where enzymatic removal of the C-peptide begins. The processing is completed in the secretory granules, which bud off from the Golgi, travel to, and fuse with, the plasma membrane thus releasing the mature hormone.

Glucose stimulates de novo insulin biosynthesis by increasing transcription, mRNA stability, translation, and protein processing. Glucose also rapidly stimulates the release of pre-stored insulin. While glucose and non-glucose secretagogues can ultimately work through a final common pathway involving alterations in K⁺ and Ca⁺⁺ channel activity and increases in intracellular Ca⁺⁺ (Prentki et al. (1987) Physiol. Rev. 67:1185; Turk et al., (1987) Prog. Lipid Res. 26:125), the biochemical events leading from changes in the levels of a particular fuel to insulin secretion are initially diverse. In the case of glucose, transport into the β-cell and metabolism of this sugar appear to be required for secretion, leading to the hypothesis that its specific stimulatory effect is mediated by, and proportional to, its flux rate through glycolysis and related pathways (Ashcroft, (1980) Diabetologia 18:5; Hedeskov, (1980) Physiol. Rev. 60:442; Meglasson et al., (1986) Diabetes/Metabolism Rev. 2:163; Prentki et al., (1987) Physiol. Rev. 67:1185; Turk et al., (1987) Prog. Lipid Res. 26:125; Malaisse et al., (1990) Biochem Soc. Trans. 18:107). Strong support for this view comes from the finding that non-metabolizable analogs of glucose such as 3-O-methyl or 2-deoxy glucose fail to stimulate insulin release (Ashcroft, (1980) Diabetologia 18:5; Meglasson et al., (1986) Diabetes/Metabolism Rev. 2:163).

There has been a focus by the medical and research communities on the development of new therapeutic approaches for diabetics. Significant effort has been devoted to the strategy of islet or pancreas fragment transplantation as a means for permanent insulin replacement (Lacy et al., (1986) Ann. Rev. Med. 37:33). However, this approach has been hampered by the difficulties associated with obtaining tissue, as well as the finding that transplanted islets are recognized and destroyed by the same autoimmune mechanism responsible for destruction of the patient's original islet β-cells.

Treatment for diabetes is still centered around self-injection of insulin once or twice daily. Both recombinant and non-recombinant methods are currently employed for the industrial production of human insulin for therapeutic use. Recombinant methods generally include the expression of recombinant proinsulin in bacteria or yeast, followed by chemical treatment of the proinsulin to ensure correct disulfide bond linkages between the A and B chains of the mature insulin molecule. The proinsulin produced by microorganisms is processed to insulin by the addition of proteolytic enzymes. Thereafter, the mature insulin peptide must be purified away from the bacterial or yeast proteins, as well as from the added proteases. The bacterial procedure involves 40 distinct steps. Non-recombinant methods typically include the purification of pig insulin from freshly isolated porcine pancreas or pancreatic islets. Each of the above methods suffers from the drawback of being technically difficult and laborious. The latter method is further complicated by the fact that the pancreas is a complex proteinaceous tissue with high levels of active proteases that can degrade insulin, thereby rendering it inactive.

Brooks et al., (1999) Proc Natl. Acad. Sci. USA 96:1129, evaluated LDH isoforms in mitochondria of rat liver and heart tissue by electrophoresis and electron microscopy. These investigators suggested that the mitochondria play a role in cellular lactate oxidation.

Previous reports have indicated that over-expression of LDH impairs IS response (Alcazar et al., (2000) Biochem. Soc. 352:373; Ainscow et al., (2000) Diabetes 49:1149; Zhao et al., (1998) FEBS Lett. 430:213), whereas Ishihara et al., (1999) J. Clin. Invest. 104:1621, found no effect on glucose stimulated IS in INS-1 cells overexpressing LDH. These results may be attributed in part to the fact that the cells employed in these studies do not exhibit a robust glucose-stimulated IS. For example, INS-1 cells are very variable in their performance, depending upon the length of time the cells have been in culture, probably explained by the recent finding that they are comprised of a heterogeneous population of subclones, only some of which are glucose-responsive (Hohmeier et al., (2000) Diabetes 49:424).

Nucleic acid and amino acid sequences encoding LDH have been described (see, e.g., GenBank Accession No. X03753 (mouse; A form); GenBank Accession No. NM_{—}010699 (mouse; A form); Y00309 (mouse; A form); Fukasawa et al., (1987) Genetics 116:99 (mouse; A form); Kayoko et al., (1986) Biochem J. 235:435 (mouse; A form); Li et.al., (1985) Eur. J. Biochem. 149:215 (mouse; A isoform); Akai et al., (1985) Int. J. Biochem. 17:645 (mouse; A form); GenBank Accession No. NM_{—}017025 (rat; A form); U.S. Pat. No. 6,057,141 (chicken; B form); Hirota et al., (1990) Nucl. Acids Res. 18:6432 (chicken; A form); GenBank Accession No. NM_{—}005566 (human; A form); GenBank Accession No. NM_{—}002300 (human; B form); U.S. Pat. No. 6,503,743 (human); U.S. Pat. No. 6,429,006 and Ishiguro et al., (1991) Gene 91:281 (bovine; A form); GenBank Accession No. AF226154 and U.S. Pat. No. 6,268,189 (Rhizopus oryzae; A form); GenBank Accession No. M22305 (B. megaterium); GenBank Accession No. M19396 (B. stearothermophylus). None of these publications, however, has suggested the presence of a mitochondrial localized form of LDH.

There is a need in the art for improved therapeutic agents and drug targets for the treatment of diabetes.

### SUMMARY OF THE INVENTION

To gain further insight into the role of mitochondrial metabolism of pyruvate in the regulation of IS by carbohydrate fuels, a recombinant adenovirus was used to overexpress lactate dehydrogenase A (LDH_{A}) in the β-cell line, 832/13, on the assumption that overexpression of this enzyme would divert pyruvate away from its mitochondrial metabolic pathways. The 832/13 line is a highly differentiated model of β-cell function provided as a subclone of the rat insulinoma cell line INS-1 (Hohmeier et al., (2000) Diabetes 49:424). Surprisingly, the present inventors have discovered that LDH overexpression potentiates insulin secretion (IS) in response to both glucose and pyruvate. The inventors believe that their results are different from those of previous studies in which overexpression of LDH was found to have no effect or to inhibit glucose-stimulated IS because the inventors used the highly glucose-responsive 832/13 cell line. LDH overexpressing cells exhibit increased lactate output, but with no change in glycolytic flux (glucose usage) relative to control cells. In addition, the LDH inhibitor, oxamate, causes a large decrease in lactate production and glucose-stimulated IS. Taken together, these data suggest that LDH overexpression enhances IS despite lesser availability of glucose-derived pyruvate for mitochondrial metabolism, and imply a central role of LDH and lactate in regulation of IS. Interestingly, lactate itself is a poor secretagogue in 832/13 cells relative to glucose or pyruvate, but overexpression of LDH allows lactate to become as effective as the other two fuels.

While not wishing to be bound by any particular theory of the invention, the inventors propose a model involving a compartmentalized form of LDH, including but not limited to an intramitochondrial form. In this model, pyruvate can enter the mitochondria as pyruvate, as lactate, or as two separate pools of pyruvate, the second pool having been produced by the compartmentalized form of LDH. In the absence of LDH overexpression and with lactate as the secretagogue, flux via the first pyruvate pool is insufficient to achieve IS. However, when LDH activity increases, the flux through this pathway is increased. When glucose and pyruvate act as secretagogues, entry via lactate or the second pyruvate pool pathway is limiting but can be enhanced by LDH overexpression. In support of this model, described herein is the cloning of a novel form of LDH and the effects of its overexpression on IS.

Moreover, while not wishing to limit the invention to any particular mechanism of action, the inventors propose that the observed effects of LDH overexpression may be related to the broader effects of pyruvate cycling on IS. ¹³C-isotopomer analysis has previously been applied to a unique set of cell lines derived from rat INS-1 cells which demonstrated a wide range of glucose-stimulated IS from weak to robust (Lu et al., (2002) Proc. Nat. Acad. Sci. USA 99:2708). These investigations found that pyruvate carboxylase-catalyzed pyruvate cycling, but not the fractional contribution of glucose to acetyl-CoA formation (pyruvate dehydrogenase-catalyzed pyruvate metabolism) was correlated with the level of glucose-responsiveness of the various cell lines. Moreover, phenylacetic acid impaired, whereas malate in the form of methyl ester (which is cell-permeable) potentiated, glucose-stimulated IS in the glucose-responsive clones in direct correlation with changes in pyruvate-catalyzed cycling of pyruvate. These results suggested that exchange of pyruvate with TCA cycle intermediates, rather than oxidation of pyruvate via acetyl-CoA, correlates with glucose-stimulated IS.

The current studies indicate that lactate plays an important function in pyruvate cycling and underscore the importance of the mitochondrial pyruvate pool in mediating glucose-stimulated IS. Thus, the present invention points to a broad array of approaches for altering fuel-stimulated IS by impacting pyruvate cycling, cytoplasmic and/or mitochondrial pyruvate pools, lactate and/or pyruvate flux from the cytoplasm into the mitochondria, the conversion of lactate to pyruvate within the mitochondria or at another compartmentalized location, the concentration of NADH in the cytoplasm and, conversely, the concentration of NAD+ in the mitochondria, and the like.

Accordingly, as one aspect, the present invention provides a method of enhancing fuel-stimulated IS (e.g., glucose-stimulated IS) in a subject, comprising administering to the subject a compound that modulates LDH activity in an amount effective to enhance fuel-stimulated IS. LDH activity can be modulated in the cytoplasm and/or in the mitochondria.

Further provided is a method of enhancing fuel-stimulated IS (e.g., glucose-stimulated IS) in a subject, comprising administering to the subject a compound that enhances the net flux of lactate from the cytoplasm into the mitochondria, enhances the net flux of pyruvate from the cytoplasm into the mitochondria, or both, wherein the compound is administered in an amount effective to enhance fuel-stimulated insulin secretion.

As still a further aspect, the invention provides a method of enhancing fuel-stimulated IS (e.g., glucose-stimulated IS) in a subject, comprising administering to the subject a compound that enhances conversion of lactate to pyruvate within the mitochondria, and/or increases the pool of pyruvate within the mitochondria available for pyruvate cycling, where the compound is administered in an amount effective to enhance fuel-stimulated IS.

As yet another aspect, the invention provides a method of enhancing fuel-stimulated IS (e.g., glucose-stimulated IS) in a subject, comprising administering to the subject a compound that increases the concentration of NADH in the cytoplasm, wherein the compound is administered in an amount effective to enhance fuel-stimulated IS.

As a further aspect, the invention provides a method of enhancing fuel-stimulated IS (e.g., glucose-stimulated IS) in a subject, comprising administering to the subject a compound that increases the concentration of NAD+ in the mitochondria, wherein the compound is administered in an amount effective to enhance fuel-stimulated IS.

As another aspect, the invention provides screening assays to identify compounds that bind to LDH, modulate LDH activity and/or modulate IS (e.g., fuel-stimulated or glucose-stimulated IS) or produce any other desired end-point. The assays can be cell-based or cell-free or, alternatively, can be carried out in a transgenic non-human animal expressing an LDH transgene.

In one representative embodiment, the invention provides a method of identifying a compound that modulates (i.e., enhances or inhibits) fuel-stimulated IS (e.g., glucose-stimulated IS), comprising: contacting a LDH polypeptide with a test compound; and detecting whether the test compound binds to and/or modulates the activity of the LDH polypeptide, thereby identifying a compound that modulates fuel-stimulated insulin secretion.

As another embodiment, the invention provides a method of identifying a compound that modulates fuel-stimulated IS (e.g., glucose stimulated IS), comprising: introducing a test compound into a cell that comprises LDH polypeptide; and detecting whether the compound modulates LDH activity in the cell, thereby identifying a compound that modulates fuel-stimulated insulin secretion. The cell can further comprise an isolated nucleic acid encoding LDH.

As yet another embodiment, the invention provides a method of identifying a compound that modulates fuel-stimulated insulin secretion (e.g., glucose-stimulated IS), comprising: introducing a test compound into a cell that is capable of producing and secreting insulin, and which comprises an isolated nucleic acid encoding LDH that is expressed to produce LDH polypeptide; and detecting the modulation of fuel-stimulated insulin secretion in the cell, thereby identifying a compound that modulates fuel-stimulated insulin secretion.

As a further aspect, the invention provides a method of identifying a compound that modulates fuel-stimulated IS (e.g., glucose-stimulated IS) comprising (a) introducing a test compound into a cell that is capable of producing and secreting insulin and detecting modulation of fuel-stimulated insulin secretion, and (b) contacting the test compound with LDH polypeptide (in a cell-free or cell-based system) and detecting whether the test compound binds to and/or modulates LDH activity. In particular embodiments, the step of introducing the test compound into the cell and detecting modulation of fuel-stimulated insulin secretion is carried out prior to the step of contacting the test compound with LDH polypeptide. In other embodiments, the step of contacting the test compound with LDH polypeptide is carried out prior to the step of introducing the compound into a cell and detecting modulation of fuel-stimulated insulin secretion. The cell can optionally contain an isolated nucleic acid encoding LDH.

As still a further aspect, the present invention provides a transgenic non-human animal (e.g., a transgenic non-human mammal) comprising an isolated nucleic acid encoding LDH operably associated with a transcriptional control element functional in pancreatic islet β-cells, wherein the isolated nucleic acid is stably incorporated into and expressed in pancreatic islet β-cells of the non-human animal. In illustrative embodiments, the transgenic non-human animal is a transgenic mouse and is optionally a mouse model for diabetes, obesity or other glucose intolerant states.

The non-human transgenic animal can be used according to the present invention in a method of screening a compound for modulation (e.g., enhancement or inhibition) of fuel-stimulated insulin secretion (e.g., glucose-stimulated insulin secretion), comprising: administering a test compound to the transgenic non-human animal; and detecting whether the test compound modulates fuel-stimulated insulin secretion in the transgenic non-human animal.

Also provided are compounds that are identified by the screening methods of the invention.

As a further aspect, the present invention provides a method of enhancing fuel-stimulated IS (e.g., glucose stimulated IS) in a subject comprising, administering to the subject an isolated nucleic acid encoding LDH in an amount effective to enhance fuel-stimulated IS. In particular embodiments, the subject is a subject with diabetes mellitus (non-insulin dependent or insulin dependent diabetes mellitus), a subject with impaired glucose tolerance, or an obese subject. The isolated nucleic acid can encode an LDH A, LDH B, or LDH C isoform (or a combination thereof). Likewise the LDH can be a cytoplasmic or mitochondrial localized (or otherwise compartmentalized, e.g., in the endoplasmic reticulum or membrane-bound within the cytoplasm) form. In particular embodiments, the isolated nucleic acid can be administered with any viral or non-viral vector or delivery system known in the art.

As a further aspect, the invention provides a method of treating non-insulin dependent diabetes mellitus comprising, administering to a subject diagnosed with non-insulin dependent diabetes mellitus an isolated nucleic acid encoding LDH in a therapeutically effective amount.

As yet another aspect, the invention provides an isolated nucleic acid encoding a compartmentalized (e.g., mitochondrial) lactate dehydrogenase subunit (A, B or C isoform). The isolated nucleic acid can be DNA (including cDNAs), RNA, or chimeras of DNA and RNA, and can further comprise nucleotide base analogs and derivatives. In some embodiments of the invention, the isolated nucleic acid encodes an mitochondrial signal peptide (e.g., at the N-terminus).

In representative embodiments, the isolated nucleic acid comprises a nucleotide sequence selected from the group consisting of:
(a) a nucleotide sequence selected from the group consisting of the nucleotide sequence of SEQ ID NO:3, SEQ ID NO:24 and SEQ ID NO:26;
(b) a nucleotide sequence that hybridizes to a nucleotide sequence selected from the group consisting of the nucleotide sequence of SEQ ID NO:3, SEQ ID NO:24 and SEQ ID NO:26 or its complementary nucleotide sequence under stringent conditions, wherein the nucleotide sequence encodes a functional LDH_{A}; and
(c) a nucleotide sequence encoding an amino acid sequence encoded by the nucleotide sequences of (a) and (b), but which has a different nucleotide sequence than the nucleotide sequences of (a) and (b) due to the degeneracy of the genetic code or the presence of non-translated nucleotide sequences.

Further, as another aspect, the invention provides an isolated compartmentalized (e.g., mitochondrial) LDH polypeptide (e.g., A, B or C isoform). The polypeptide can be a monomer or it can be complexed with other subunits (e.g., to form homo or hetero-tetramers).

Further provided are cultured cells comprising the isolated nucleic acids and polypeptides of the invention for use in cell-based screening assays.

As still another aspect, the invention provides the use of isolated nucleic acids encoding LDH in the manufacture of a medicament for the treatment of diabetes (insulin-dependent or non-insulin dependent) or other glucose intolerant states.

These and other aspects of the present invention will be discussed in more detail in the description of the invention set forth below.

### BRIEF DESCRIPTION OF THE DRAWINGS

FIG. 1. The metabolic pathways involving pyruvate in pancreatic β-cells. Pyruvate has been shown to have three different destinies in the β-cell. First, pyruvate can be converted to lactate in the cytosol, a process catalyzed by lactate dehydrogenase (LDH). LDH activity is known to be very low in β-cells. Thus, the vast majority of glucose-derived pyruvate will enter the mitochondria and either be converted into acetyl-CoA for subsequent oxidation in the TCA cycle, or will be carboxylated to oxaloacetate by pyruvate carboxylase. In β-cells, the amount of glucose-derived pyruvate that enters mitochondria metabolism by carboxylation almost equals that which enters by decarboxylation. It has been proposed that pyruvate carboxylase-catalyzed anaplerotic influx of pyruvate is linked to an export of NADPH equivalents from the mitochondria which in turn might be important for IS regulation. Furthermore, by ¹³C NMR isotopomer analyses evidence has been obtained for a direct correlation between pyruvate cycling (substrate flux though pyruvate carboxylase and back to pyruvate) and insulin secretion in the INS-1 derived cell line 832/13.

FIG. 2A. Nucleotide sequence for rat LDH_{A}cDNA (Accession Number NM_{—}017025, SEQ ID NO:1).

FIG. 2B. The 332 amino acid sequence of rat LDH_{A} (SEQ ID NO:2). The translation corresponds to nucleotides 104-1609 of SEQ ID NO:1 (FIG. 2A).

FIG. 3. Stimulation of insulin secretion by lactate, pyruvate, and glucose in 832/13 cells. Confluent, insulin secreting 832/13 cells were stimulated for 2 hours with various concentrations of secretagogues as indicated in the figure, and IS was determined by radioimmunoassay of the cell supernatant.

FIG. 4. The effect of rat LDH_{A} overexpression on glucose, pyruvate, and lactate-stimulated insulin secretion in 832/13 cells. 832/13 cells were treated with adenovirus expressing rat LDH_{A} (AdLDH) and beta-galactosidase (Adbetagal), respectively. The LDH activity in cell extracts was monitored by the decrease in absorbance at 340 nm (insert) and the insulin response to glucose, pyruvate, and lactate was examined by radioimmunoassay.

FIG. 5. The effect of rat LDH_{A} overexpression on glucose usage in 832/13 cells. In order to compare glycolytic flux in cells overexpressing rat LDH_{A} and beta-galactosidase, respectively, cells were incubated for 2 hours in the presence of [3-³H]glucose. After deproteination, supernatants were transferred to microcentrifuge tubes and placed overnight at 50° C. in tightly closed scintillation vials. Glucose usage was determined from the level of ³H₂O released to the scintillation tubes.

FIG. 6. The effect of rat LDH_{A} overexpression on lactate production in 832/13 cells. Lactate production from insulin secreting 832/13 cells treated with AdLDH was compared with output from Adbetagal virus treated control cells using a lactate oxidase/peroxidase linked assay combined with A₅₄₀ nm measurement. The increased lactate production in LDH overexpressing cells indicates that the overexpressed enzyme functions in the direction of lactate production.

FIG. 7A. The effect of the LDH inhibitor oxamate on lactate output in 832/13 cells. Cells were incubated with ¹³C labeled glucose for 4 hours in the presence or absence of oxamate.

FIG. 7B. The effect of the LDH inhibitor oxamate on insulin secretion in 832/13 cells. Cells were incubated with ¹³C labeled glucose for 4 hours in the presence or absence of oxamate.

FIG. 7C. The effect of the LDH inhibitor oxamate on pyruvate cycling in 832/13 cells. Cells were incubated with ¹³C labeled glucose for 4 hours in the presence or absence of oxamate and pyruvate cycling was determined by ¹³C NMR analysis as-described in Lu et al., (2000) Proc. Nat. Acad. Sci. USA 99:2708.

FIG. 7D. The effect of the LDH inhibitor oxamate on insulin secretion in islet β-cells. Cells were incubated with ¹³C labeled glucose for 4 hours in the presence or absence of oxamate.

FIG. 8 depicts a model involving a mitochondrial form of LDH_{A}. Pyruvate can enter the mitochondria in two ways, either as pyruvate, or as lactate which in turn is converted to pyruvate inside the mitochondria. It is proposed that both these entry pathways need to be active in order to obtain IS.

FIG. 9 depicts the effect of glycerol kinase overexpression in 832/13 cells on insulin excretion, lactate output and pyruvate cycling.

FIG. 10. The organization of the mouse cytoplasmic LDH_{A} gene. The gene (12.9 kb) for cytoplasmic LDH_{A} consists of 8 exons (in black) with the translational start site present in the second exon that gives rise to a protein of 332 amino acids. An alternative exon (diagonally striped, SEQ ID NO:18) between exon 1 (SEQ ID NO:19) and exon 2 has been identified and contains an alternative start site (capital letters). Transcripts which have this alternative exon spliced to the 5′ end of exon 2 will give rise to a LDH protein with an additional 29 amino acids (SEQ ID NO:20) at the N-terminal end that have the features of a mitochondrial targeting signal.

FIG. 11 shows the peptide sequence of the 29 amino acid leader sequence of the putative mit-LDH_{A} from mouse (SEQ ID NO:20), rat (SEQ ID NO:21) and human (SEQ ID NO:22), along with the consensus peptide sequence (SEQ ID NO:23).

FIG. 12A shows the nucleic acid sequence (SEQ ID NO:3) for the cDNA clone of the rat mitochondrial form of LDH_{A}.

FIG. 12B shows the amino acid sequence (SEQ ID NO:4) for the rat mitochondrial form of LDH_{A} from the translation of nucleotides 101-1186 of SEQ ID NO:3.

FIG. 12C shows the nucleic acid sequence (SEQ ID NO:24) for the cDNA clone of the mouse mitochondrial form of LDH_{A}.

FIG. 12D shows the amino acid sequence (SEQ ID NO:25) for the mouse mitochondrial form of LDH_{A} from the translation of nucleotides 111-1193 of SEQ ID NO:24.

FIG. 12E shows the nucleic acid sequence (SEQ ID NO:26) for the cDNA clone of the human mitochondrial form of LDH_{A}.

FIG. 12F shows the amino acid sequence (SEQ ID NO:27) for the human mitochondrial form of LDH_{A} from the translation of nucleotides 111-1193 of SEQ ID NO:26.

FIGS. 13A-D shows the alignment of rat (SEQ ID NO:3), mouse (SEQ ID NO:24), and human (SEQ ID NO:26) mitochondrial LDH_{A} cDNA sequences.

FIG. 14. The effect of mitLDH_{A} and LDH_{A} on fuel-mediated IS in the 832/13 cells. The inset depicts the activity of LDH for each experiment.

### DETAILED DESCRIPTION OF THE PREFERRED EMBODIMENTS

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Except as otherwise indicated, standard methods can be used for the production of viral and non-viral vectors, manipulation of nucleic acid sequences, production of transformed cells, and the like according to the present invention. Such techniques are known to those skilled in the art. See, e.g., SAMBROOK et al., MOLECULAR CLONING: A LABORATORY MANUAL 2nd Ed. (Cold Spring Harbor, NY, 1989); F. M. AUSUBEL et al. CURRENT PROTOCOLS IN MOLECULAR BIOLOGY (Green Publishing Associates, Inc. and John Wiley & Sons, Inc., New York).

I. Definitions.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

The term “modulate,” “modulates” or “modulation” refers to enhancement (e.g., an increase) or inhibition (e.g., a reduction) in the specified activity.

Similarly, the term “modulate LDH activity,” “modulates LDH activity” or “modulation of LDH activity” refers to an enhancement (e.g., an increase) or inhibition (e.g., a reduction) in LDH enzymatic activity (for example, as measured by conversation of pyruvate to lactate). In representative embodiments, modulation of LDH activity within the cells of the pancreatic islets of Langerhans is of interest (e.g., in the islet β-cells). Those skilled in the art will appreciate that in the practice of particular embodiments of the invention, it will not be possible or will be difficult to measure LDH activity within the pancreatic islets of a live animal. Thus, according to the invention, a determination as to whether a compound modulates LDH activity can be made in vivo in an intact animal or a tissue removed from the animal; alternatively, modulation of LDH enzymatic activity can be detected in isolated pancreatic islet β-cells, cell lines derived from pancreatic islet β-cells (e.g., insulinoma cells), or any other suitable cell in culture.

The term “overexpress,” “overexpresses” or “overexpression” as used herein in connection with isolated nucleic acids encoding an LDH transgene refers to expression that results in higher levels of LDH polypeptide than exist in the cell in its native (untransformed) state. Overexpression of LDH can result in levels that are 25%, 50%, 100%, 200%, 500%, 1000%, 2000% or higher in the cell. Further, the LDH can be introduced into a cell that does not produce the specified form of LDH (e.g., A isoform or mitochondrial LDH) encoded by the transgene or does so only at negligible levels.

As used herein, the term “diabetes” is used interchangeably with the term “diabetes-mellitus.” The terms “diabetes” and “diabetes mellitus” are intended to encompass both insulin dependent and non-insulin dependent (Type I and Type II, respectively) diabetes mellitus, unless one condition or the other is specifically indicated.

The term “insulin secretion” (IS) as used herein refers to secretion of insulin from a cell, e.g., into the systemic circulation or cell culture medium, and will typically refer to secretion of insulin from pancreatic islet β-cells, although it can also refer to secretion from cells which have been engineered to express a recombinant insulin (for example, artificial β-cells). Insulin secretion can be assessed directly, for example, by measuring plasma insulin concentrations using art-known methods such as radioimmunoassay. Alternatively, insulin secretion can be indirectly evaluated by measuring, for example, changes in plasma glucose concentrations (or glucose concentrations in cell culture medium).

“Fuel-stimulated insulin secretion” refers to the commencement or enhancement of insulin secretion in response to an elevation in the extracellular concentration of carbohydrates (e.g., glucose, glycerol, glyceraldehyde), amino acids and/or fatty acids. In particular embodiments, fuel-stimulated insulin secretion results in an increase of at least about 2-fold, 3-fold, 4-fold, 5-fold, 6-fold, 8-fold, 10-fold, twelve-fold, or even fifteen-fold or more in insulin secretion over baseline levels in the presence of a sufficiently high concentration of fuels. In other words, the level of fuel-stimulated insulin secretion can be dependent on the concentration of the fuel(s), but the maximal elevation in insulin secretion is at least about 2-fold, 3-fold, 4-fold, 5-fold, 6-fold, 8-fold, 10-fold, twelve-fold, or even fifteen-fold or more over baseline levels. Alternatively, fuel-stimulated insulin secretion can be commenced in a cell or subject that did not previously have any detectable fuel-stimulated insulin secretion (or only negligible levels).

“Glucose-stimulated insulin secretion” refers to the compensatory secretion of insulin in response to an elevation in serum glucose (e.g., following a meal or a glucose challenge) or, in the case of cultured cells, to an elevation of glucose concentration in the cell culture medium. In particular embodiments, glucose-stimulated insulin secretion results in an increase of at least about 2-fold, 3-fold, 4-fold, 5-fold, 6-fold, 8-fold, 10-fold, twelve-fold, or even fifteen-fold or more in insulin secretion over baseline levels in the presence of a sufficiently high concentration of glucose. In other words, the level of glucose-stimulated insulin secretion can be dependent on the glucose concentration, but the maximal elevation in insulin secretion is at least about 2-fold, 3-fold, 4-fold, 5-fold, 6-fold, 8-fold, 10-fold, twelve-fold, or even fifteen-fold or more over baseline levels. Alternatively, glucose-stimulated insulin secretion can be commenced in a cell or subject that did not previously have any detectable glucose-stimulated insulin secretion (or only negligible levels).

The term “enhance,” “enhances,” “enhancing” or “enhancement” with respect to insulin secretion refers to an increase in insulin secretion (e.g., at least about a 1.5-fold, 2-fold, 3-fold, 4-fold, 5-fold, 6-fold, 8-fold, 10-fold, twelve-fold, or even fifteen-fold or more increase), for example, in response to elevated glucose concentrations. Alternatively, these terms can refer to commencing insulin secretion in a cell or subject that did not previously have any detectable insulin secretion. In particular embodiments, fuel-stimulated IS is enhanced in a cell or subject comprising an isolated nucleic acid encoding LDH according to the invention as compared with the level of fuel-stimulated IS in a comparable cell in the absence of the isolated nucleic acid overexpressing LDH.

By “providing fuel-simulated [or glucose-stimulated] insulin secreting capability” to a subject, it is meant that fuel-stimulated (or glucose-stimulated) insulin secreting capability is enhanced as described above. Thus, fuel-stimulated (or glucose-stimulated) insulin secretion can be commenced in a subject that did not previously have detectable fuel-stimulated (or glucose-stimulated) insulin secretion or can be increased above previous levels.

The term “glucose tolerance” refers to a state in which there is proper functioning of the homeostatic mechanisms by which insulin is secreted in response to an elevation in serum glucose concentrations. Impairment in this system results in transient hyperglycemia as the organism is unable to maintain normoglycemia following a glucose load (for example, a carbohydrate containing meal) because of insufficient secretion of insulin from the islet β-cells or because of insensitivity of target tissues to circulating insulin.

“An improvement in glucose tolerance” is a level of amelioration of glucose tolerance that provides some clinical benefit to the subject. Glucose tolerance can be assessed by methods known in the art, such as for example, the oral glucose tolerance test which monitors serum glucose concentrations following an oral glucose challenge. In particular embodiments, an “improvement in glucose tolerance” can result in normalization of fasting or baseline serum glucose concentrations, a reduction in maximal serum glucose concentrations, and/or an improved temporal response to a glucose challenge.

A “transgenic” non-human animal is a non-human animal that comprises a foreign nucleic acid incorporated into the genetic makeup of the animal, such as for example, by stable integration into the genome or by stable maintenance of an episome (e.g., derived from EBV).

A “therapeutically effective” amount as used herein is an amount that provides some improvement or benefit to the subject. Alternatively stated, a “therapeutically-effective” amount is an amount that provides some alleviation, mitigation, or decrease in at least one clinical symptom of glucose intolerance or diabetes in the subject (e.g., improved glucose tolerance, enhanced glucose-stimulated insulin secretion, and the like) as is well-known in the art. Those skilled in the art will appreciate that the therapeutic effects need not be complete or curative, as long as some benefit is provided to the subject.

By the terms “treating” or “treatment of,” it is intended that the severity of the patient's condition is reduced or at least partially improved or modified and that some alleviation, mitigation or decrease in at least one clinical symptom is achieved.

As used herein, a “vector” or “delivery vector” can be a viral or non-viral vector that is used to deliver a nucleic acid to a cell, tissue or subject.

A “recombinant” vector or delivery vector refers to a viral or non-viral vector that comprises one or more heterologous nucleotide sequences (i.e., transgenes), e.g., two, three, four, five or more heterologous nucleotide sequences. Generally, the recombinant vectors of the invention encode LDH, but can also comprise one or more additional heterologous sequences.

As used herein, the term “viral vector” or “viral delivery vector” can refer to a virus particle that functions as a nucleic acid delivery vehicle, and which comprises the vector genome packaged within a virion. Alternatively, these terms can be used to refer to the vector genome when used as a nucleic acid delivery vehicle in the absence of the virion.

A viral “vector genome” refers to the viral genomic DNA or RNA, in either its naturally occurring or modified form. A “recombinant vector genome” is a viral genome (e.g., vDNA) that comprises one or more heterologous nucleotide sequence(s).

A “heterologous nucleotide sequence” will typically be a sequence that is not naturally-occurring in the vector. Alternatively, a heterologous nucleotide sequence can refer to a sequence that is placed into a non-naturally occurring environment (e.g., by association with a promoter with which it is not naturally associated).

By “infectious,” as used herein, it is meant that a virus can enter a cell by natural transduction mechanisms and express viral genes and/or nucleic acids (including transgenes). Alternatively, an “infectious” virus is one that can enter the cell by other mechanisms and express the coding sequences carried by the viral genome therein. As one illustrative example, the vector can enter a target cell by expressing a ligand or binding protein for a cell-surface receptor in the virion or by using an antibody(ies) directed against molecules on the cell-surface followed by internalization of the complex.

As used herein, the term “polypeptide” encompasses both peptides and proteins, unless indicated otherwise.

A “fusion polypeptide” is a polypeptide produced when two heterologous nucleotide sequences or fragments thereof coding for two (or more) different polypeptides not found fused together in nature are fused together in the correct translational reading frame. Illustrative fusion polypeptides include fusions of LDH (or a portion thereof) to all or a portion of glutathione-S-transferase, maltose-binding protein, or a reporter protein (e.g., Green Fluorescent Protein, β-glucuronidase, β-galactosidase, luciferase, etc.).

As used herein, a “functional” polypeptide is one that retains at least one biological activity normally associated with that polypeptide. Preferably, a “functional” polypeptide retains all of the activities possessed by the unmodified peptide. By “retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits essentially no detectable biological activity normally associated with the polypeptide (e.g., at most, only an insignificant amount, e.g., less than about 10% or even 5%).

A “recombinant” nucleic acid is one that has been created using genetic engineering techniques.

A “recombinant polypeptide” is one that is produced from a recombinant nucleic acid.

As used herein, an “isolated” nucleic acid (e.g., an “isolated DNA” or an “isolated vector genome”) means a nucleic acid separated or substantially free from at least some of the other components of the naturally occurring organism or virus, such as for example, the cell or viral structural components or other polypeptides or nucleic acids commonly found associated with the nucleic acid.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w).

By the term “express,” “expresses” or “expression” of a nucleic acid coding sequence, in particular a LDH coding sequence, it is meant that the sequence is transcribed, and optionally, translated. Typically, according to the present invention, transcription and translation of the coding sequence will result in production of LDH polypeptide.

II. Lactate Dehydrogenase.

Mammalian LDH is a tetrameric enzyme composed of A and B subunits, also known as M (muscle) and H (heart) forms, respectively. There are five isozymes of LDH enzyme resulting from the assembly of homotetramers (AAAA or BBBB) or heterotetramers (ABBB, AABB, AAAB) of the A and B isoforms. The B isoform predominates in heart muscle and facilitates the aerobic oxidation of pyruvate. The A subunit predominates in skeletal muscle and liver and is primarily implicated with anaerobic metabolism and pyruvate reduction to lactate. To date, only the A form has been identified in islet β-cells. Another LDH isoform, LDH_{C}, has been identified in spermatozoa.

LDH shares structural similarities with other NAD-binding enzymes. The polypeptide chain of each subunit folds into two clearly separated domains. The two domains have different functions, and appear to each comprise a separate module. One of the domains (domain 1) binds to the coenzyme, NAD/NADH, and the second (domain 2) binds the substrate (e.g., pyruvate or lactate) and also provides the amino acid residues that are involved in catalysis. The coenzyme-binding domain is in the amino-terminal portion of the polypeptide. The active site of the enzyme is the cleft or “vacuole” that is formed between the two domains. The coenzyme-binding site on the one domain and the substrate-binding site on the other are oriented so that the C4 position of the nicotinamide ring is in close proximity to the hydrogen atom to be transferred between the substrate and coenzyme (Introduction to Protein Structure. 1991. Branden and Tooze (eds). Garland Publishing, Inc., New York. pg. 144).

The LDH inhibitor, oxamate, binds at the active site of the enzyme with the NADH coenzyme. Structural analysis of the LDH_{A}-oxamate-NADH complex indicates that NADH binds at the top end of the central parallel beta sheet and the mobile active site loop above it in domain 1. Domain 2 forms the other side of the active site, around and behind the oxamate. There is also an amino-terminal tail that is non-compact in the monomer but wraps around another subunit in the intact tetramer. The active site cleft includes Asn 140, with its side chain nitrogen forming a hydrogen bond with oxygens on both the oxamate and the nicotinamide ring.

The mobile active site loop (residues 94-120) forms as a result of charge changes in the vacuole. It is believed that the amino acid residues Arg 109, Arg 171, His 195 and Asn 140 are important for enzymatic activity. Cortes et al., (1992) Protein Sci 1:892, have reported that formation of the active site vacuole is dependent on the ionizing groups within the vacuole having the same total overall charge as is present in the wild type enzyme complex with NAD+ and lactate (incorporated by reference herein in its entirety for its teachings of the effects of amino acid substitutions and charge on LDH enzymatic activity). Substitution of an Asp residue for Asn 140 resulted in a 10-fold increase in the Km for pyruvate for each unit increase in pH over pH 4.5 up to pH 9. These investigators concluded that the anion of Asp 140 was completely inactive and that it bound pyruvate with a Km that is more than 1,000 times greater than the Km of the neutral protonated Asp 140. Further, this report indicates that the active site vacuole is only sufficiently large to accept substrates up to C4 in the presence of bound coenzyme.

The term “lactate dehydrogenase” or “LDH” as used herein, is intended to be construed broadly and encompasses the A, B and C forms of LDH as well as cytoplasmic, mitochondrial, or otherwise compartmentalized forms of the enzyme (e.g., in the endoplasmic reticulum or membrane-bound within the cytoplasm). The term “lactate dehydrogenase” or “LDH” also includes modified (e.g., mutated) LDH that retain biological function (i.e., have at least one biological activity of the native LDH protein, e.g., converting pyruvate → lactate), functional LDH fragments including truncated molecules, and functional LDH fusion polypeptides (e.g., an LDH-maltose binding protein fusion).

Generally, the functional LDH fragment forms an active site cleft and is able to bind substrate (e.g., pyruvate or lactate) and coenzyme (e.g., NAD or NADH). In representative embodiments, a functional LDH fragment comprises at least about 25, 50, 100, 150 or 200 amino acids of the full-length polypeptide. In other embodiments, the functional LDH fragment comprises domain 1 (coenzyme binding domain) and/or domain 2 (catalytic and substrate binding domain). In still other embodiments, the functional LDH fragment comprises the residues that form the mobile active site loop (amino acids 94-120).

Moreover, the term “lactate dehydrogenase” or “LDH” refers to a single subunit (e.g., A isoform) or a multimer of subunits (e.g., the mature tetrameric enzyme), or both, depending upon the context. In particular embodiments, the LDH is an LDH_{A} monomer or multimeric assembly of LDH_{A} (e.g., a homotetramer).

In illustrative embodiments, the LDH is compartmentalized. For example, the LDH can be a mitochondrial targeted LDH (e.g., to the mitochondrial matrix). By “mitochondria targeted” it is intended that intracellular processing results in a substantial portion of the nascent protein being directed to and localized in the mitochondria (for example, in the mitochondrial matrix, inner membrane and/or intermembrane space). This definition does not exclude the possibility that some or even all of the mitochondria targeted LDH is translocated or leaked into the cytoplasm where it can exert its cellular effects on IS as described herein.

The mitochondria targeted LDH can be naturally occurring or can be produced by recombinant nucleic acid techniques by engineering an LDH polypeptide to be targeted to the mitochondria (e.g., to the mitochondrial matrix) by in-frame fusion of a sequence encoding a mitochondria signal peptide (e.g., of about 10 to about 50 or 100 amino acids or more in length), typically at the amino terminus, as known in the art. Suitable signal sequences for localizing LDH to a mitochondrial compartment of interest can be derived from the precursors of proteins that normally reside in that mitochondrial compartment. Known mitochondrial targeting sequences vary in length, from about 10-70 residues, and the most common sequence similarity among them is the predominance, all along their length, of basic residues, hydroxyl-containing Ser and Thr residues, and small hydrophobic residues (Proteins: Structures and Molecular Properties. Second edition. (1993) Thomas E. Creighton (ed.), W.H. Freeman and Company, New York; incorporated herein by reference in its entirety for teachings of mitochondrial targeting sequences).

Exemplary mitochondrial targeting peptides (in particular, targeting peptides that result in localization in the mitochondrial matrix) have been described in U.S. patent application Publication No. 20020151014 (MLSRLSLRLLSRYLL; SEQ ID NO:5); Whelan and Glaser, (1997) Plant Mol. Biol. 33:771-789 (providing a review of mitochondrial targeting sequences); Close, Pamela S., Cloning and Molecular Characterization of Two Nuclear Genes for Zea mays Mitochondrial Chaperonin 60, Doctoral Thesis, Iowa State University (1993) (describing a mitochondrial signal sequence from the maize chaperonin 60 gene; MYRAAASLASKARQAGSSSAARQVGSRLAWSRNY; SEQ ID NO:6). Other mitochondria targeting sequences that have been reported include the sequence MLSLRQSIRFFPATRTLCSSRYLL (SEQ ID NO:7) and the mitochondria targeting sequences discussed in Proteins: Structures and Molecular Properties. Second edition. (1993) Thomas E. Creighton (ed.), W.H. Freeman and Company, New York. pg. 72 (for example, MLRTSSLFTRRVQPSLFSRNILRLQST, SEQ ID NO:8; MLSLRQSIRFFKPATRT, SEQ ID NO:9; MFSNLSKRWAQRTLSKSFYST (SEQ ID NO:10; MKSFITRNKT, SEQ ID NO:11).

Alternatively, the mitochondrial targeting sequence can be wholly or partially synthetic.

In still other embodiments, the LDH is a cytoplasmic LDH. By “cytoplasmic” LDH, it is intended that intracellular processing results in a substantial portion of the newly-synthesized LDH protein being directed to and localized in the cytoplasm of the cell.

In still other embodiments, overexpression of the LDH results in enhancement of mitochondrial pyruvate concentrations.

Any LDH polypeptide or LDH-encoding nucleic acid known in the art can be used according to the present invention. The LDH polypeptide or LDH-encoding nucleic acid can be derived from bacterial, yeast, fungal, plant or animal (e.g., insect, avian (e.g., chicken), mammalian (e.g., rat, mouse, bovine, porcine, ovine, caprine, equine, feline, canine, lagomorph, simian, human and the like) sources.

Exemplary LDH polypeptides and LDH-encoding nucleic acids, include but are not limited to, those disclosed in: GenBank Accession No. X03753 (mouse; A form); GenBank Accession No. NM_{—}010699 (mouse; A form); Y00309 (mouse; A form); Fukasawa et al., (1987) Genetics 116:99 (mouse; A form); Kayoko et al., (1986) Biochem J. 235:435 (mouse; A form); Li et al., (1985) Eur. J. Biochem. 149:215 (mouse; A isoform); Akai et al., (1985) Int. J. Biochem. 17:645 (mouse; A form); GenBank Accession No. NM_{—}017025 (rat; A form); U.S. Pat. No. 6,057,141 (chicken; B form); Hirota et al., (1990) Nucl. Acids Res. 18:6432 (chicken; A form); GenBank Accession No. NM_{—}005566 (human; A form); GenBank Accession No. NM_{—}002300 (human; B form); U.S. Pat. No. 6,503,743 (human); U.S. Pat. No. 6,429,006 and Ishiguro et al., (1991) Gene 91:281 (bovine; A form); GenBank Accession No. AF226154 and U.S. Pat. No. 6,268,189 (Rhizopus oryzae; A form); GenBank Accession No. M22305 (B. megaterium); GenBank Accession No. M19396 (B. stearothermophylus); the disclosures of which are incorporated herein by reference in their entireties for their teachings of LDH coding sequences and proteins.

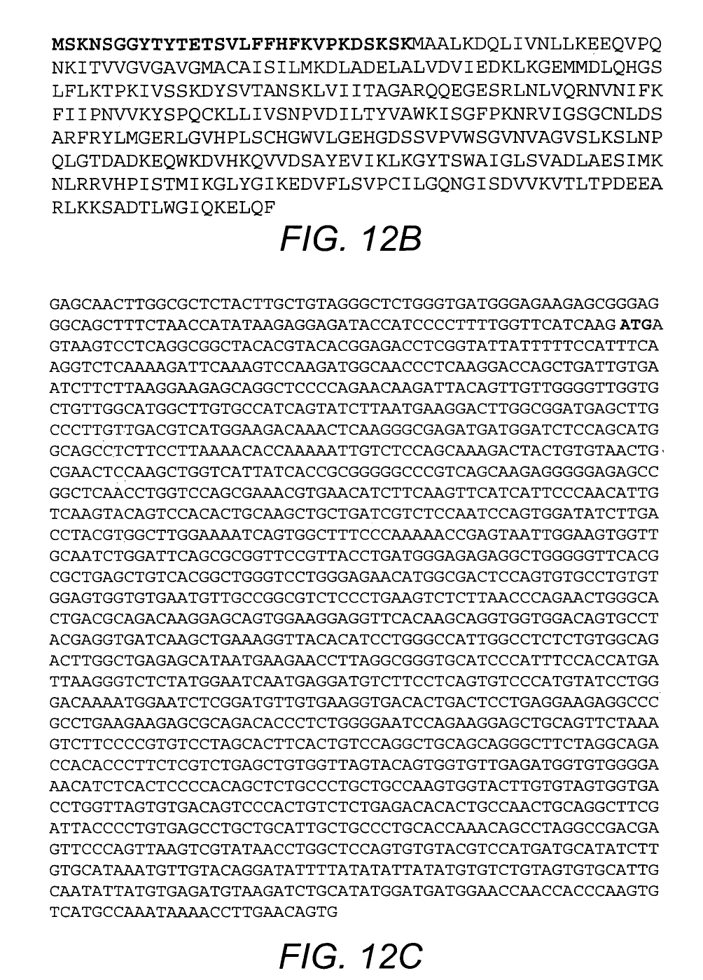

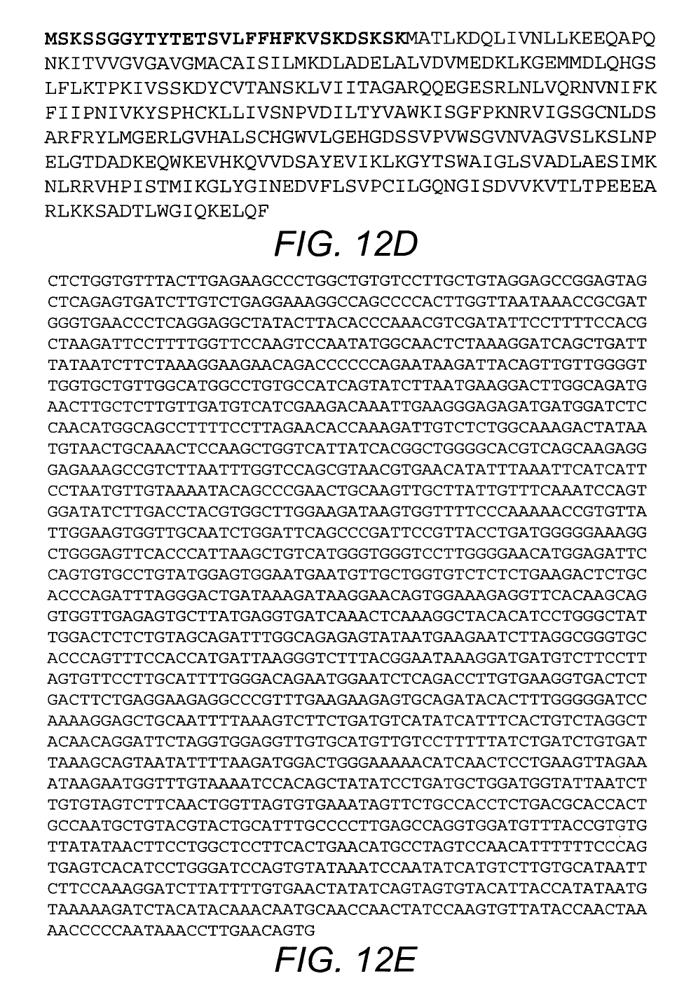

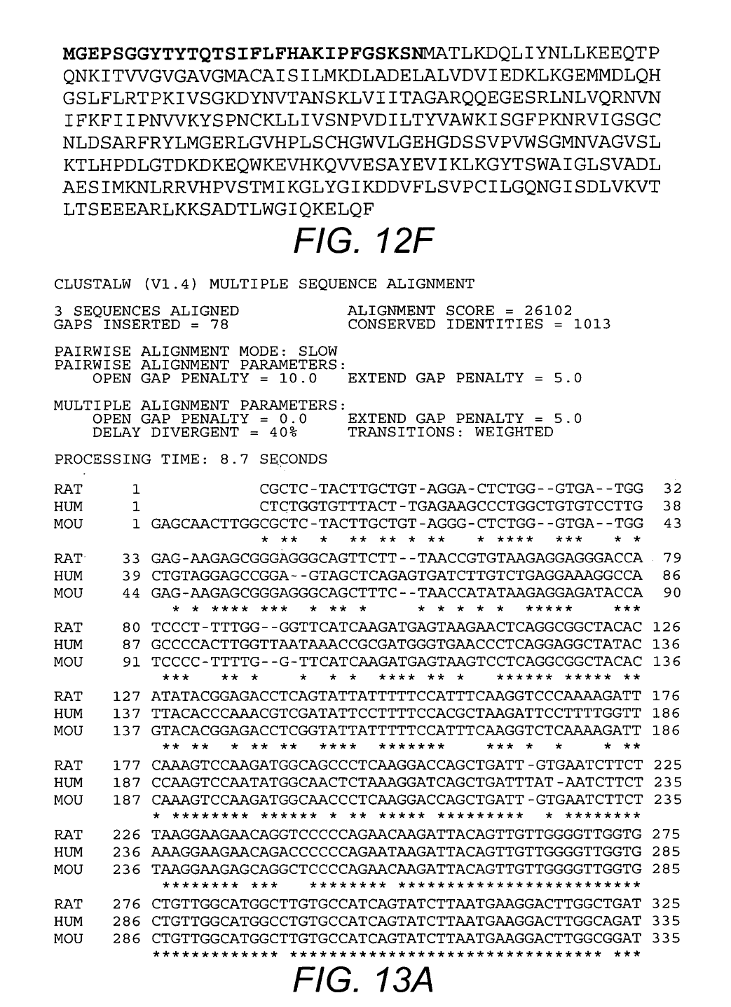

Representative cDNA and amino acid sequences of a rat mitochondrial (or otherwise compartmentalized) LDH_{A} are shown in SEQ ID NO:3 and SEQ ID NO:4, respectively (FIGS. 12A and 12B). Exemplary nucleotide and amino acid sequences of the mitochondrial LDH_{A} from mouse (SEQ ID NO:24 and SEQ ID NO:25, respectively) and human mitochondrial LDH_{A} (SEQ ID NO:26 and SEQ ID NO:27, respectively) are also disclosed (FIGS. 12C-12F). Other mitochondrial LDH encompassed by the present invention are described in more detail below.

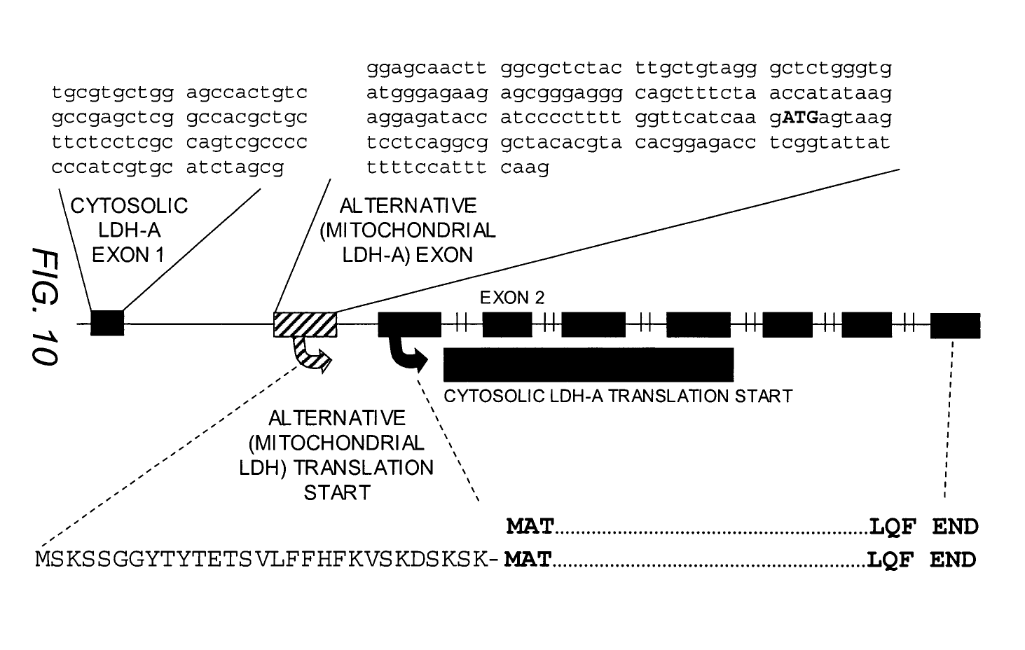

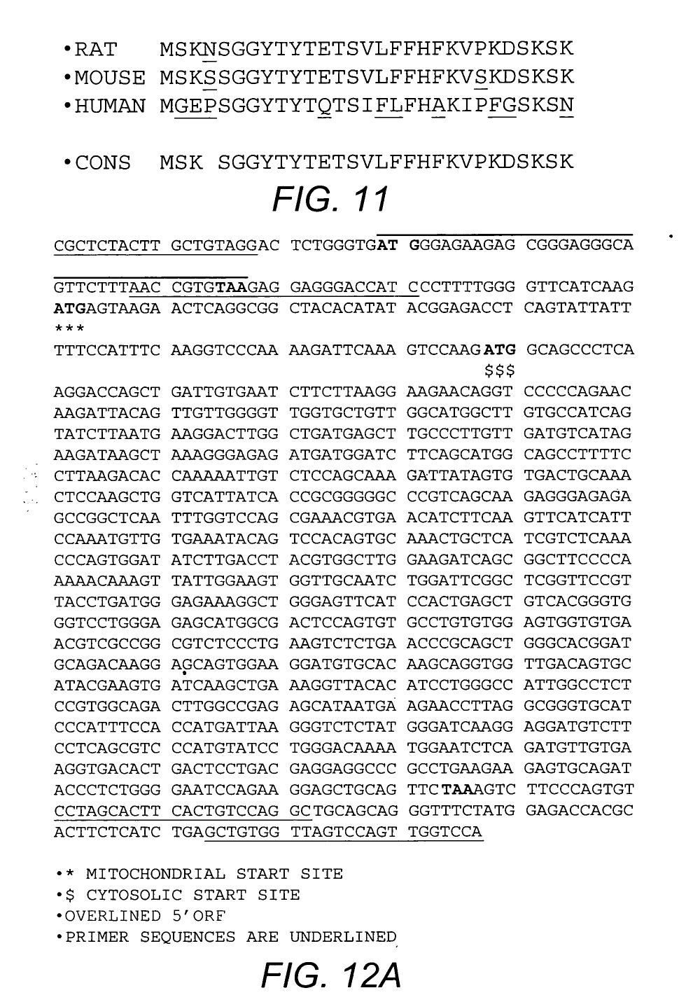

The present inventors have identified a new exon in the mouse and human LDH_{A} genes (FIG. 10) that contains an alternative translational start site and encodes an LDH_{A} isoform having a putative amino terminal mitochondrial signal peptide, which amino terminal sequences share characteristics with known mitochondrial signal peptides. The inventors have further cloned a rat liver cDNA encoding a LDH_{A} having an additional 29 amino acids at the amino terminus (SEQ ID NO:21; FIG. 10 and FIG. 11) as compared with the cytosolic form, and which shares a high degree of amino acid sequence similarity with the amino terminal sequences of the rat and human LDH.

The isolated nucleic acids of the invention can encode mitochondrial LDH_{A}, LDH_{B}, and/or LDH_{C} isoform. Likewise, the isolated nucleic acid can encode a mitochondrial LDH from any species, as described above.

In representative embodiments of the invention, the isolated nucleic acid encoding the mitochondrial LDH will hybridize to the nucleic acid sequences encoding LDH specifically disclosed herein (i.e., SEQ ID NO:1, SEQ ID NO:3, SEQ ID NO:24 or SEQ ID NO:26) under standard conditions as known by those skilled in the art and encode a functional mitochondrial LDH as defined herein. Such sequences are intended to encompass fragments of the full-length LDH coding sequence that hybridize to the nucleic acid sequences encoding LDH specifically disclosed herein. Generally, fragments of the full-length LDH coding sequence encompassed by the present invention will encode a functional LDH polypeptide (as defined above) of at least about 25, 50, 100, 150 or 200 amino acids or longer having the specified properties (e.g., as a mitochondrial targeted LDH).

To illustrate, hybridization of such sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively) to the sequences specifically disclosed herein. See, e.g., Sambrook et al., Molecular Cloning, A Laboratory Manual (2d Ed. 1989) (Cold Spring Harbor Laboratory).

Alternatively stated, isolated nucleic acids encoding mitochondrial LDH of the invention have at least about 60%, 70%, 80%, 90%, 95%, 97%, 98% or higher sequence similarity with the isolated nucleic acid sequences specifically disclosed herein (or fragments thereof, as defined above) and encode a functional mitochondrial LDH as defined herein.

It will be appreciated by those skilled in the art that there can be variability in the nucleic acids that encode the mitochondrial LDH of the present invention due to the degeneracy of the genetic code. The degeneracy of the genetic code, which allows different nucleic acid sequences to code for the same polypeptide, is well known in the literature (see Table 1).

TABLE 1

| | |
|---|---|
| Amino Acids | Codons |

| | | | |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC ACT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Further variation in the nucleic acid sequence can be introduced by the presence (or absence) of non-translated sequences, such as intronic sequences and 5′ and 3′ untranslated sequences.

Moreover, the isolated nucleic acids of the invention encompass those nucleic acids encoding mitochondrial LDH polypeptides that have at least about 60%, 70%, 80%, 90%, 95%, 97%, 98% or higher amino acid sequence similarity with the polypeptide sequences specifically disclosed herein (or fragments thereof) and further encode a functional mitochondrial LDH as defined herein.

As is known in the art, a number of different programs can be used to identify whether a nucleic acid or polypeptide has sequence identity or similarity to a known sequence. Sequence identity and/or similarity can be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values can be adjusted to increase sensitivity.

An additional useful algorithm is gapped BLAST as reported by Altschul et al., (1997) Nucleic Acids Res. 25, 3389-3402.

A percentage amino acid sequence identity value can be determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment can include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

To modify the LDH amino acid sequences specifically disclosed herein or otherwise known in the art, amino acid substitutions can be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding LDH.

In making amino acid substitutions, the hydropathic index of amino acids can be considered. The importance of the hydropathic amino acid index in conferring interactive biologic function on a protein is generally understood in the art (see, Kyte and Doolittle, (1982) J. Mol. Biol. 157:105; incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are:
isoleucine (+4.5); valine (+4.2); leucine (+3.8); phenylalanine (+2.8);
cysteine/cystine (+2.5); methionine (+1.9); alanine (+1.8); glycine (−0.4);
threonine (−0.7); serine (−0.8); tryptophan (−0.9); tyrosine (-1.3); proline (−1.6);
histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Isolated nucleic acids of this invention include RNA, DNA (including cDNAs) and chimeras thereof. The isolated nucleic acids can further comprise modified nucleotides or nucleotide analogs.

The isolated nucleic acids encoding LDH can be associated with appropriate expression control sequences, e.g., transcription/translation control signals and polyadenylation signals.

It will be appreciated that a variety of promoter/enhancer elements can be used depending on the level and tissue-specific expression desired. The promoter can be constitutive or inducible (e.g., the metalothionein promoter or a hormone inducible promoter), depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest. In particular embodiments, the promoter functions in pancreatic islet β-cells or in cells that can be used to express nucleic acids encoding LDH for the purposes of large-scale protein production. Likewise, the promoter can be “specific” for these cells and tissues (i.e., only show significant activity in the specific cell or tissue type), for example, the insulin promoter for islet β-cells; the prolactin or growth hormone promoters for anterior pituitary cells.

To illustrate, a LDH coding sequence can be operatively associated with a cytomegalovirus (CMV) major immediate-early promoter, an albumin promoter, an Elongation Factor 1-α (EF1-α) promoter, a PγK promoter, a MFG promoter, a Rous sarcoma virus promoter, an insulin promoter, or a glyceraldehyde-3-phosphate promoter.

Moreover, specific initiation signals are generally required for efficient translation of inserted protein coding sequences. These translational control sequences, which can include the ATG initiation codon and adjacent sequences, can be of a variety of origins, both natural and synthetic.

In embodiments of the invention wherein the isolated nucleic acid encoding LDH comprises an additional sequence to be transcribed, the transcriptional units can be operatively associated with separate promoters or with a single upstream promoter and one or more downstream internal ribosome entry site (IRES) sequences (e.g., the picornavirus EMC IRES sequence).

The isolated nucleic acids encoding LDH can be incorporated into a vector, e.g., for the purposes of cloning or other laboratory manipulations, recombinant protein production, or gene delivery. Exemplary vectors include bacterial artificial chromosomes, cosmids, yeast artificial chromosomes, phage, plasmids, lipid vectors and viral vectors (described in more detail below). Expression vectors are described in more detail in Section VII below. Nucleic acid delivery vectors are more specifically described in Section V.

The present invention further provides cells comprising the isolated nucleic acids encoding LDH for use in the screening methods and large-scale protein production methods of the invention (e.g., insulin or LDH is produced and collected from the cells and, optionally, purified). In one particular embodiment, the invention provides a cultured cell comprising an isolated nucleic acid encoding LDH as described above for use in a screening assay for identifying a compound that enhances fuel-stimulated (e.g., glucose-stimulated) IS. Also provided is a cell in vivo produced by a method comprising administering an isolated nucleic acid encoding LDH to a subject in a therapeutically effective amount. Such methods exclude cell-based therapeutic methods, i.e., ex vivo genetic manipulation of cells that are implanted into a subject. Thus, according to this embodiment of the invention, the cell is not an isolated cell that has been modified to express an isolated nucleic acid encoding LDH, which can then be administered to a subject. The cells of the present invention explicitly exclude isolated cells for used in cell-based therapeutic methods.

III. Screening Methods.

The finding that LDH overexpression potentiates fuel-stimulated IS points to LDH as a new drug target for identifying compounds for diabetes therapy. Accordingly, in one aspect, the present invention provides methods of identifying a compound or compounds that bind to and/or modulate LDH and/or modulate IS. Any desired end-point can be detected, e.g., binding to LDH, modulation of LDH activity, and/or modulation of IS (particularly, fuel-stimulate IS, more particularly, glucose-stimulated IS). Alternatively, the detected end-point can be pyruvate cycling, cytoplasmic and/or mitochondrial pyruvate pools, lactate and/or pyruvate flux from the cytoplasm into the mitochondria, the conversion of lactate to pyruvate within the mitochondria, the conversion of pyruvate to lactate in the cytoplasm, the concentration of NADH in the cytoplasm, the concentration of NAD+ in the mitochondria, and the like.

Any compound of interest can be screened according to the present invention. Suitable test compounds include organic and inorganic molecules. Suitable organic molecules can include, but are not limited to, polypeptides (including antibodies and Fab′ fragments), carbohydrates, lipids, coenzymes, and nucleic acid molecules (including DNA, RNA and chimerics and analogs thereof). In particular embodiments, the compound is an antisense nucleic acid, an interfering RNA (RNAi), or a ribozyme that inhibits production of LDH polypeptide or any other molecule the inhibition of which results in modulation of LDH activity and/or IS activity.

Further, the methods of the invention can be practiced to screen a compound library, e.g., a combinatorial chemical compound library, a polypeptide library, a cDNA library, a library of antisense nucleic acids, and the like, or an arrayed collection of compounds such as polypeptide and nucleic acid arrays.

In one representative embodiment, the invention provides methods of screening test compounds to identify a test compound that binds to LDH polypeptide (either monomers, tetramers, or functional fragments of monomers, as described below). Compounds that are identified as binding to LDH can be subject to further screening using the methods described herein (e.g., for modulation of LDH activity and/or modulation of fuel-stimulated IS, including glucose-stimulated IS) or other suitable techniques.

Also provided are methods of screening compounds to identify those that modulate LDH activity (e.g., cytoplasmic and/or mitochondrial LDH activity). The term “modulate” is intended to refer to compounds that enhance (e.g., increase) or inhibit (e.g., reduce) LDH activity. Methods of assessing LDH enzyme activity in animal tissues, cells, or cell-free preparations are standard in the art, for example, spectrophotometric methods that follow the conversion of pyruvate to lactate in the presence of NADH at 340 nm. Compounds that are identified as modulators of LDH activity can optionally be further screened using the methods described herein (e.g., for binding to LDH and/or modulation of fuel-stimulated IS, including glucose-stimulated IS) or other suitable techniques. The compound can directly interact with LDH and thereby modulate its activity. Alternatively, the compound can interact with any other polypeptide, nucleic acid or other molecule as long as the interaction results in a modulation of LDH activity.

In still a further embodiment, compounds are screened to identify those which compete for binding to LDH by a known LDH inhibitor (described in the next section, e.g., oxamate or a salt thereof). According to this embodiment, either the known inhibitor or the test compound can be modified for detection (e.g., with a radiolabel of fluorescent label) and competition for binding to LDH by the test compound can thus be detected.

As another aspect, the invention provides a method of screening compounds for modulation of IS (e.g., fuel-stimulated IS, more particularly, glucose-stimulated IS). In one representative embodiment, the method comprises contacting LDH polypeptide with a test compound; and detecting whether the test compound binds to LDH and/or modulates the activity of the LDH. In another exemplary embodiment, the method comprises introducing a test compound into a cell that comprises LDH polypeptide; and detecting whether the compound binds to LDH and/or modulates LDH activity in the cell. The LDH can be endogenously produced in the cell. Alternatively or additionally, the cell can be modified to comprise an isolated nucleic acid encoding LDH.

In another illustrative embodiment, the method comprises introducing a test compound into a cell that is capable of producing and secreting insulin and which comprises LDH polypeptide; and detecting whether the compound modulates IS (e.g., fuel-stimulated IS or glucose-stimulated IS) by the cell. The LDH can be endogenously produced by the cell. Alternatively or additionally, the cell can be modified to comprise an isolated nucleic acid encoding, and optionally overexpressing, LDH.

In still other representative embodiments, the invention provides a method of identifying a compound that modulates fuel-stimulated IS (e.g., glucose-stimulated IS) comprising introducing a test compound into a cell that is capable of producing and secreting insulin and detecting modulation of fuel-stimulated insulin secretion. The method further comprises contacting the test compound with LDH polypeptide and detecting whether the test compound binds to and/or modulates LDH activity. Thus, this embodiment comprises multiple screening/characterization steps. In particular embodiments, the step of introducing the test compound into the cell and detecting modulation of fuel-stimulated insulin secretion is carried out prior to the step of contacting the test compound with LDH polypeptide to determine whether it binds to and/or modulates LDH activity. In other embodiments, the step of contacting the test compound with LDH polypeptide is carried out prior to the step of introducing the compound into a cell and detecting modulation of fuel-stimulated insulin secretion. The cell can optionally contain an isolated nucleic acid encoding LDH.

The screening assays of the invention can be carried out with LDH multimers (e.g., assembled LDH tetramers), a monomeric LDH subunit, or a functional fragment of a monomeric LDH subunit. The LDH can be an A form monomer, a B form monomer, a C form monomer, or homotetramers or heterotetramers thereof. Moreover, the LDH can be a mitochondrial or cytoplasmic form.

An isolated nucleic acid can be provided that encodes one or more LDH subunits. Alternatively, more than one isolated nucleic acid encoding one or more LDH subunits can be provided. Likewise, the cell can produce one or more LDH subunits from endogenous (i.e., native) nucleic acid sequences.

The screening assay can be a cell-based or cell-free assay. Further, the LDH (as described above) can be free in solution, affixed to a solid support, expressed on a cell surface, or located within a cell.

With respect to cell-free binding assays, test compounds can be synthesized or otherwise affixed to a solid substrate, such as plastic pins, glass slides, plastic wells, and the like. For example, the test compounds can be immobilized utilizing conjugation of biotin and streptavidin by techniques well known in the art. The test compounds can be contacted with LDH and washed. Bound LDH can be detected using standard techniques in the art (e.g., by radioactive or fluorescence labeling of the LDH, by ELISA methods, and the like).

Alternatively, the LDH target can be immobilized to a solid substrate and the test compounds contacted with the bound LDH. Identifying those test compounds that bind to and/or modulate LDH can be carried out with routine techniques. For example, the test compounds can be immobilized utilizing conjugation of biotin and streptavidin by techniques well known in the art. As another illustrative example, antibodies reactive with LDH can be bound to the wells of the plate, and the LDH trapped in the wells by antibody conjugation. Preparations of test compounds can be incubated in the LDH-presenting wells and the amount of complex trapped in the well can be quantitated.

In another representative embodiment, a fusion protein can be provided which comprises a domain that facilitates binding of the protein to a matrix. For example, glutathione-S-transferase fusion proteins can be adsorbed onto glutathione sepharose beads (Sigma Chemical, St. Louis, Mo.) or glutathione derivatized microtitre plates,.which are then combined with cell lysates (e.g., ³⁵S-labeled) and the test compound, and the mixture incubated under conditions conducive to complex formation (e.g., at physiological conditions for salt and pH). Following incubation, the beads are washed to remove any unbound label, and the matrix immobilized and radiolabel detected directly, or in the supernatant after the complexes are dissociated. Alternatively, the complexes can be dissociated from the matrix, separated by SDS-PAGE, and the level of LDH found in the bead fraction quantitated from the gel using standard electrophoretic techniques.

Another technique for compound screening provides for high throughput screening of compounds having suitable binding affinity to the polypeptide of interest, as described in published PCT application WO 84/03564. In this method, as applied to LDH, a large number of different small test compounds are synthesized on a solid substrate, such as plastic pins or some other surface. The test compounds are reacted with LDH and washed. Bound LDH is then detected by methods well known in the art. Purified LDH can also be coated directly onto plates for use in the aforementioned drug screening techniques. Alternatively, non-neutralizing antibodies can be used to capture the peptide and immobilize it on a solid support.

With respect to cell-based assays, any suitable cell can be used including bacteria, yeast, insect cells (e.g., with a baculovirus expression system), avian cells, mammalian cells, or plant cells. With particular respect to mammalian cells, screening can advantageously be carried out with insulinoma cells (e.g., INS-1 cells including their derivatives such as the highly glucose-responsive line 832/13, HIT-T15 cells, RINr1046-38 cells, MSL-G2 cells, β-cells expressing T-antigen, typically referred to as TC cells, including bTC-3 and bTC-6 cells, MIN6 cells) or primary β-cells, “artificial β-cells” (e.g., as described by U.S. Pat. No. 5,744,327), fibroblasts, primary hepatocytes, hepatoma cell lines, and CHO cells. In particular embodiments, the cells exhibits fuel-responsive (e.g., glucose-responsive) IS.

The screening assay can be used to detect compounds that bind to or modulate the activity of native LDH (e.g., LDH that is normally produced by the cell). Alternatively, the cell can be modified to express a recombinant LDH. According to this embodiment, the cell can be transiently or stably transformed with the nucleic acid encoding LDH, but is preferably stably transformed, for example, by stable integration into the genome of the organism or by expression from a stably maintained episome (e.g., Epstein Barr Virus derived episomes).

In a cell-based assay, the compound to be screened can interact directly with the LDH polypeptide or coding sequence (i.e., bind to it) and modulate the activity thereof. Alternatively, the compound to be screened can interact with the substrate of the LDH enzyme (e.g., pyruvate or lactate) and/or any other cellular component, interaction with which results in an indirect modulation of LDH activity. LDH activity can be modulated by effecting a change in the enzymatic activity and/or stability of the polypeptide. Alternatively, the compound can be one that modulates LDH activity at the nucleic acid level. To illustrate, the compound can modulate transcription of the LDH gene (or transgene), modulate the accumulation of LDH mRNA (e.g., by affecting the rate of transcription and/or turnover of the mRNA), and/or modulate the rate and/or amount of translation of the LDH mRNA transcript.

As a further type of cell-based binding assay, LDH can be used as a “bait protein” in a two-hybrid or three-hybrid assay (see, e.g., U.S. Pat. No. 5,283,317; Zervos et al., (1993) Cell 72:223-232; Madura et al., (1993) J. Biol. Chem. 268:12046-12054; Bartel et al., (1993) Biotechniques 14:920-924; Iwabuchi et al., (1993) Oncogene 8:1693-1696; and PCT publication WO94/10300), to identify other polypeptides that bind to or interact with LDH.

The two-hybrid system is based on the modular nature of many transcription factors, which consist of separable DNA-binding and activation domains. Briefly, the assay utilizes two different nucleic acid constructs. In one construct, the nucleic acid that encodes LDH is fused to a nucleic acid encoding the DNA binding domain of a known transcription factor (e.g., GAL-4). In the other construct, a nucleic acid sequence, optionally from a library of DNA sequences, that encodes an unidentified protein (“prey” or “sample”) is fused to a nucleic acid that codes for the activation domain of the known transcription factor. If the “bait” and the “prey” proteins are able to interact in vivo, forming a complex, the DNA-binding and activation domains of the transcription factor are brought into close proximity. This proximity allows transcription of a reporter sequence (e.g., LacZ), which is operably linked to a transcriptional regulatory site responsive to the transcription factor. Expression of the reporter can be detected and cell colonies containing the functional transcription factor can be isolated and used to obtain the nucleic acid encoding the polypeptide that exhibited binding to LDH.

As another cell-based assay, the invention provides a method of screening a compound for modulation of IS (e.g., fuel-stimulated IS, or glucose-stimulated IS) in the cell. In particular embodiments, the cell comprises an isolated nucleic acid encoding LDH. According to this embodiment, it is preferred that the isolated nucleic acid encoding LDH is stably incorporated into the cell (i.e., by stable integration into the genome of the organism or by expression from a stably maintained episome such as Epstein Barr Virus derived episomes). Methods of detecting IS and fuel-stimulated IS are known in the art. For example, insulin secretion can be readily assessed by standard techniques by collecting the medium in which the cells have been cultured and measuring the level of insulin secreted into the medium using known techniques, such as radioimmunoassay, radioreceptor assay, ELISA, and the like.

Screening assays can also be carried out in vivo in animals. Thus, as still a further aspect, the invention provides a transgenic non-human animal comprising an isolated nucleic acid encoding LDH, which can be produced according to methods well-known in the art. The transgenic non-human animal can be any species, including avians and non-human mammals. According to this aspect of the invention, suitable non-human mammals include mice, rats, rabbits, guinea pigs, goats, sheep, pigs and cattle. Mammalian models for glucose intolerance, obesity, and diabetes can also be used (e.g., STZ diabetic mice, ob/ob mice). Suitable avians include chickens, ducks, geese, quail, turkeys and pheasants.

The nucleic acid encoding LDH is stably incorporated into cells within the transgenic animal (typically, by stable integration into the genome or by stably maintained episomal constructs). It is not necessary that every cell contain the transgene, and the animal can be a chimera of modified and unmodified cells, as long as a sufficient number of pancreatic islet β-cells comprise and express the LDH transgene so that the animal is a useful screening tool (e.g., so that administration of compounds that modulate LDH activity give rise to a detectable modulation in LDH activity and/or IS).

In particular embodiments, it is desirable that the LDH transgene be operably associated with a promoter or other transcriptional regulatory element that is functional in islet β-cells or is even specific to islet β-cells (i.e., only shows insignificant activity in initiating transcription in other cell types). The insulin promoter is an illustrative islet β-cell specific promoter.

One exemplary method of using the transgenic non-human animals of the invention for in vivo screening of compounds that modulate IS (e.g., fuel-stimulated IS or glucose-stimulated IS) and/or LDH activity comprises administering a test compound to a transgenic non-human animal (e.g., a mammal such as a mouse) stably comprising an isolated nucleic acid encoding LDH, administering a test compound to the transgenic non-human animal, and detecting whether the test compound modulates IS and/or LDH activity. For example, glucose-stimulated IS can be directly assessed by measuring plasma insulin levels in response to a glucose challenge or can be indirectly assessed by evaluating the rate at which plasma glucose levels are normalized following a glucose challenge (e.g., by detecting serum glucose following an oral glucose tolerance test).

Methods of making transgenic animals are known in the art. DNA constructs can be introduced into the germ line of an avian or mammal to make a transgenic animal. For example, one or several copies of the construct can be incorporated into the genome of an embryo by standard transgenic techniques.

In an exemplary embodiment, a transgenic non-human animal is produced by introducing a transgene into the germ line of the non-human animal. Transgenes can be introduced into embryonal target cells at various developmental stages. Different methods are used depending on the stage of development of the embryonal target cell. The specific line(s) of any animal used should, if possible, be selected for general good health, good embryo yields, good pronuclear visibility in the embryo, and good reproductive fitness.

Introduction of the transgene into the embryo can be accomplished by any of a variety of means known in the art such as microinjection, electroporation, lipofection or a viral vector. For example, the transgene can be introduced into a mammal by microinjection of the construct into the pronuclei of the fertilized mammalian egg(s) to cause one or more copies of the construct to be retained in the cells of the developing mammal(s). Following introduction of the transgenic construct into the fertilized egg, the egg can be incubated in vitro for varying amounts of time, or reimplanted into the surrogate host, or both. One common method is to incubate the embryos in vitro for about 1-7 days, depending on the species, and then reimplant them into the surrogate host.

The progeny of the transgenically manipulated embryos can be tested for the presence of the construct (e.g., by Southern blot analysis) of a segment of tissue. An embryo having one or more copies of the exogenous cloned construct stably integrated into the genome can be used to establish a permanent transgenic animal line carrying the transgenically added construct.

Transgenically altered animals can be assayed after birth for the incorporation of the construct into the genome of the offspring. This can be done by hybridizing a probe corresponding to the DNA sequence coding for the polypeptide or a segment thereof onto chromosomal material from the progeny. Those progeny found to contain at least one copy of the construct in their genome are grown to maturity.

Methods of producing transgenic avians are also known in the art, see, e.g., U.S. Pat. No. 5,162,215.

IV. Compounds and Methods for Modulating Lactate Dehydrogenase Activity.

The present invention further provides a method of enhancing IS, in particular, fuel-stimulated IS (e.g., glucose-stimulated IS), comprising administering to the subject or a cell in vivo a compound that modulates LDH activity, thereby enhancing IS, the compound administered in an amount effective to enhance IS or glucose-stimulated IS. The compound can interact directly with LDH or the coding sequence for LDH to modulate the activity thereof. Alternatively, the compound can interact with any other polypeptide, nucleic acid or other molecule if such interaction results in a modulation of LDH activity.

In another embodiment, the invention provides a method of enhancing fuel-stimulated IS (e.g., glucose-stimulated IS) comprising introducing into a cultured cell a compound that modulates LDH activity and thereby enhancing IS, in an amount effective to enhance fuel-stimulated IS, subject to the proviso that the compound is not a nucleic acid encoding LDH and/or the cell is not used for ex vivo genetic modification for a cell-based therapy). In other embodiments, the method is subject to the proviso that the compound is not a nucleic acid encoding a mitochondrial LDH. Also provided are cells, including cultured cells, produced by the foregoing methods for use in the screening methods and recombinant protein production methods described herein. The cells of the present invention do not encompass isolated cells for use in cell-based therapeutic methods.

In still other embodiments, the invention provides a method of enhancing fuel-stimulated IS (e.g., glucose-stimulated IS) comprising administering to a subject or a cell in vivo a compound that enhances mitochondrial pyruvate pools available for pyruvate cycling, enhances lactate and/or pyruvate flux from the cytoplasm into the mitochondria, enhances the conversion of lactate to pyruvate within the mitochondria, enhances the conversion of pyruvate to lactate in the cytoplasm, enhances the concentration of NADH in the cytoplasm and/or enhances the concentration of NAD+ in the mitochondria, thereby enhancing IS, wherein the compound is administered in an amount effective to enhance fuel-stimulated IS.

As still another embodiment, the invention provides a method of enhancing fuel-stimulated IS (e.g., glucose-stimulated IS) comprising introducing into a cultured cell a compound that enhances mitochondrial pyruvate pools available for pyruvate cycling, enhances lactate and/or pyruvate flux from the cytoplasm into the mitochondria, enhances the conversion of lactate to pyruvate within the mitochondria, enhances the conversion of pyruvate to lactate in the cytoplasm, enhances the concentration of NADH in the cytoplasm and/or enhances the concentration of NAD+ in the mitochondria, thereby enhancing IS, wherein the compound is administered in an amount effective to enhance fuel-stimulated IS, subject to the proviso that the compound is not a nucleic acid encoding LDH and/or the cell is not used for ex vivo genetic modification for a cell-based therapy. In other embodiments, the method is subject to the proviso that the compound is not a nucleic acid encoding a mitochondrial LDH.

The compounds of this invention can enhance or inhibit the activity of LDH, and can further be an LDH inactivator or an LDH activator. The term “LDH activator,” as used herein, refers to a molecule that directly binds to LDH to increase or enhance the activity thereof. The term “LDH inactivator,” as used herein, refers to a molecule that directly binds to LDH to inhibit or reduce the activity thereof.

Compounds that specifically bind to the LDH polypeptide can bind at the active site of the enzyme or elsewhere, and can alter binding of the coenzyme NADH and/or can compete for binding by known LDH inhibitors (such as oxamate).

The term “compound” as used herein is intended to be interpreted broadly and encompasses organic and inorganic molecules. Organic compounds include, but are not limited to polypeptides, lipids, carbohydrates, coenzymes and nucleic acid molecules.

Polypeptides include but are not limited to antibodies (described in more detail below) and enzymes. Nucleic acids include but are not limited to DNA, RNA and DNA-RNA chimeric molecules. Suitable RNA molecules include RNAi, antisense RNA molecules and ribozymes (all of which are described in more detail below). The nucleic,acid can further encode any polypeptide such that administration of the nucleic acid and production of the polypeptide results in a modulation of LDH activity.

Administration of nucleic acids that encode LDH to a subject for therapeutic or other purposes is described in more detail in Sections V to VII. Compounds that are known LDH inhibitors can also be administered according to the methods of the invention. In representative embodiments, the compound is selected from the group consisting of oxamate, N-isopropyl oxamate, and their pharmaceutically acceptable salts, a 1,4-dihydropyridine or a pharmaceutically acceptable salt thereof, oxalic acid or a pharmaceutically acceptable salt thereof, pyruvic acid or a pharmaceutically acceptable salt thereof, malonic acid or a pharmaceutically acceptable salt thereof, tartronic acid or a pharmaceutically acceptable salt thereof, ethylenediaminetetraacetic acid or a pharmaceutically acceptable salt thereof, iodoacetamide or a pharmaceutically acceptable salt thereof, an iodide, and a silver salt.

In other illustrative embodiments, the compound competes with a known LDH inhibitor (e.g., oxamate) for binding to LDH.

The compound can further be a compound that is identified by any of the screening methods described herein (see Section III).

The compounds of the present invention can optionally be administered in conjunction with other therapeutic agents useful in the treatment of diabetes, other glucose intolerant conditions, or obesity. For example, the compounds of the invention can be administered in conjunction with insulin therapy and/or hypoglycemic agents.

The additional therapeutic agents can optionally be administered concurrently with the compounds of the invention. As used herein, the word “concurrently” means sufficiently close in time to produce a combined effect (that is, concurrently can be simultaneously, or it can be two or more events occurring within a short time period before or after each other).

The coding sequences for LDH from a variety of species are known (see Section II above) and an antisense nucleotide sequence or nucleic acid encoding an antisense nucleotide sequence can be generated to any portion thereof in accordance with known techniques.

The term “antisense nucleotide sequence,” as used herein, refers to a nucleotide sequence that is complementary to a specified DNA or RNA sequence. Antisense RNA sequences and nucleic acids that express the same can be made in accordance with conventional techniques. See, e.g., U.S. Pat. No. 5,023,243 to Tullis; U.S. Pat. No. 5,149,797 to Pederson et al.

As illustrative examples of an antisense nucleotide sequence that can be used to carry out the invention is a nucleotide sequence that is complementary to the nucleotide sequences of SEQ ID NO:1, SEQ ID NO:3, SEQ ID NO:24 or SEQ ID NO:26 (or a portion thereof). An antisense nucleotide sequence can be designed that is specific for mitochondrial LDH, for example, by directing the antisense nucleotide sequence to the mitochondrial isoform specific sequences (e.g., see, e.g., FIG. 11; SEQ ID NO:20, SEQ ID NO:21, SEQ ID NO:22, SEQ ID NO:23).

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of LDH polypeptide (e.g., by at least about 40%, 50%, 60%, 70%, 80%, 90%, 95% or more). As is known in the art, a higher degree of sequence similarity is generally required for short antisense nucleotide sequences, whereas a greater degree of mismatched bases will be tolerated by longer antisense nucleotide sequences.

In representative embodiments of the invention, the antisense nucleotide sequence will hybridize to the nucleotide sequences encoding LDH specifically disclosed herein (e.g., SEQ ID NO:1, SEQ ID NO:3, SEQ ID NO:24 or SEQ ID NO:26 or portions thereof) and will reduce the level of LDH polypeptide production (as defined above).

For example, hybridization of such nucleotide sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively) to the nucleotide sequences specifically disclosed herein. See, e.g., Sambrook et al., Molecular Cloning, A Laboratory Manual (2d Ed. 1989) (Cold Spring Harbor Laboratory).

Alternatively stated, antisense nucleotide sequences of the invention have at least about 60%, 70%, 80%, 90%, 95%, 97%, 98% or higher sequence similarity with the complement of the LDH coding sequences specifically disclosed herein and will reduce the level of LDH polypeptide production (as defined above).

In other embodiments, the antisense nucleotide sequence can be directed against any coding sequence, the silencing of which results in a modulation of LDH activity and/or IS activity.

The length of the antisense nucleotide sequence (i.e., the number of nucleotides therein) is not critical as long as it binds selectively to the intended location and reduces transcription and/or translation of the target sequence (e.g., by at least about 40%, 50%, 60%, 70%, 80%, 90%, 95% or more), and can be determined in accordance with routine procedures. In general, the antisense nucleotide sequence will be from about eight, ten or twelve nucleotides in length up to about 20, 30, 50, 60 or 70 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleotide sequence can be produced using an expression vector into which a nucleic acid has been cloned in an antisense orientation (i.e., RNA transcribed from the inserted nucleic acid will be of an antisense orientation to a target nucleic acid of interest).

The antisense nucleotide sequences of the invention further include nucleotide sequences wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17, 9193-9204; Agrawal et al., (1990) Proc. Natl. Acad. Sci. USA 87,1401-1405; Baker et al., (1990) Nucleic Acids Res. 18, 3537-3543; Sproat et al., (1989) Nucleic Acids Res. 17, 3373-3386; Walder and Walder, (1988) Proc. Natl. Acad. Sci. USA 85, 5011-5015; incorporated by reference herein in their entireties for their teaching of methods of making antisense molecules, including those containing modified nucleotide bases).

RNA interference (RNAi) provides another approach for modulating LDH activity and/or IS activity. The RNAi can be directed against the LDH coding sequence in the cell or any other sequence that results in modulation of LDH activity and/or IS activity.

RNAi is a mechanism of post-transcriptional gene silencing in which double-stranded RNA (dsRNA) corresponding to a coding sequence of interest is introduced into a cell or an organism, resulting in degradation of the corresponding mRNA. The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8). In one embodiment, silencing can be induced in mammalian cells by enforcing endogenous expression of RNA hairpins (see Paddison et al., (2002), PNAS USA 99:1443-1448). In another embodiment, transfection of small (e.g., 21-23 nt) dsRNA specifically inhibits nucleic acid expression (reviewed in Caplen, (2002) Trends in Biotechnology 20:49-51).

The mechanism by which RNAi achieves gene silencing has been reviewed in Sharp et al, (2001) Genes Dev 15: 485-490; and Hammond et al., (2001) Nature Rev Gen 2:110-119).

RNAi technology utilizes standard molecular biology methods. To illustrate, dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Silencing effects similar to those produced by RNAi have been reported in mammalian cells with transfection of a mRNA-cDNA hybrid construct (Lin et al., (2001) Biochem Biophys Res Commun 281:639-44), providing yet another strategy for silencing a coding sequence of interest.

The compound can further by a ribozyme. Ribozymes are RNA-protein complexes that cleave nucleic acids in a site-specific fashion. Ribozymes have specific catalytic domains that possess endonuclease activity (Kim et al., (1987) Proc. Natl. Acad. Sci. USA 84:8788; Gerlach et al., (1987) Nature 328:802; Forster and Symons, (1987) Cell 49:211). For example, a large number of ribozymes accelerate phosphoester transfer reactions with a high degree of specificity, often cleaving only one of several phosphoesters in an oligonucleotide substrate (Michel and Westhof, (1990) J. Mol. Biol. 216:585; Reinhold-Hurek and Shub, (1992) Nature 357:173). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., (1991)Proc. Natl. Acad. Sci. USA 88:10591; Sarver et al., (1990) Science 247:1222; Sioud et al., (1992) J. Mol. Biol. 223:831).

A compound of the invention can further be an antibody or antibody fragment. The antibody or antibody fragment can bind to LDH (e.g., at the active site) or to any other polypeptide of interest, as long as the binding between the antibody or the antibody fragment and the target polypeptide results in modulation of LDH activity.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, or human, or can be a chimeric antibody. See, e.g., Walker et al., Molec. Immunol. 26, 403-11 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980.

Antibody fragments included within the scope of the present invention include, for example, Fab, F(ab′)2, and Fc fragments, and the corresponding fragments obtained from antibodies other than IgG. Such fragments can be produced by known techniques. For example, F(ab′)2 fragments can be produced by pepsin digestion of the antibody molecule, and Fab fragments can be generated by reducing the disulfide bridges of the F(ab′)2 fragments. Alternatively, Fab expression libraries can be constructed to allow rapid and easy identification of monoclonal Fab fragments with the desired specificity (Huse etal., (1989) Science 254, 1275-1281).

Polyclonal antibodies used to carry out the present invention can be produced by immunizing a suitable animal (e.g., rabbit, goat, etc.) with an antigen to which a monoclonal antibody to the target binds, collecting immune serum from the animal, and separating the polyclonal antibodies from the immune serum, in accordance with known procedures.

Monoclonal antibodies used to carry out the present invention can be produced in a hybridoma cell line according to the technique of Kohler and Milstein, (1975) Nature 265, 495-97. For example, a solution containing the appropriate antigen can be injected into a mouse and, after a sufficient time, the mouse sacrificed and spleen cells obtained. The spleen cells are then immortalized by fusing them with myeloma cells or with lymphoma cells, typically in the presence of polyethylene glycol, to produce hybridoma cells. The hybridoma cells are then grown in a suitable medium and the supernatant screened for monoclonal antibodies having the desired specificity. Monoclonal Fab fragments can be produced in E. coli by recombinant techniques known to those skilled in the art. See, e.g., W. Huse, (1989) Science 246, 1275-81.

Antibodies specific to the target polypeptide can also be obtained by phage display techniques known in the art.

V. Delivery Vectors.

The methods of the present invention provide a means for delivering, and optionally expressing, nucleic acids encoding LDH in a broad range of host cells, including both dividing and non-dividing cells in vitro (e.g., for large-scale recombinant protein production or for use in screening assays) or in vivo (e.g., for recombinant large-scale protein production, for creating an animal model for disease, or for therapeutic purposes). In embodiments of the invention, the nucleic acid can be expressed transiently in the target cell or the nucleic acid can be stably incorporated into the target cell, for example, by integration into the genome of the cell or by persistent expression from stably maintained episomes (e.g., derived from Epstein Barr Virus).

As one aspect, the isolated nucleic acids, vectors, methods and pharmaceutical formulations of the present invention find use in a method of administering a nucleic acid encoding LDH to a subject. In this manner, LDH can thus be produced in vivo in the subject. The subject can have a deficiency of LDH, or the production of a foreign LDH in the subject can impart some therapeutic effect. Pharmaceutical formulations and methods of delivering nucleic acids encoding LDH for therapeutic purposes are described in more detail in Section VI below.

Alternatively, an isolated nucleic acid encoding LDH can be administered to a subject so that the nucleic acid is expressed by the subject and LDH is produced and purified therefrom, i.e., as a source of recombinant LDH protein. According to this embodiment, it is preferred that the LDH is secreted into the systemic circulation or into another body fluid (e.g., milk, lymph, spinal fluid, urine) that is easily collected and from which the LDH can be further purified. As a further alternative, LDH protein can be produced in avian species and deposited in, and conveniently isolated from, egg proteins.

Likewise, LDH-encoding nucleic acids can be expressed transiently or stably in a cell culture system for the purpose of screening assays (described in Section III above) or for large-scale recombinant protein production (prokaryotic and eukaryotic expression systems are described in more detail in described in Section VII below). The cell can be a bacterial, protozoan, plant, yeast, fungus, or animal cell. Preferably, the cell is an animal cell (e.g., insect, avian or mammalian), and more preferably a mammalian cell (e.g., a fibroblast).

The methods of the present invention (as discussed above) can be carried out by introducing a nucleic acid encoding a single LDH isoform (e.g., the A isoform) into a cell or subject, or alternatively, by co-introduction of nucleic acids encoding the A, B and/or C isoforms. Co-introduction can involve introduction of a single vector encoding LDH A, B and/or C isoforms or separate vectors encoding each of the subunits.

Berman & Newgard, (1998) Biochemistry 37:4543, found that overexpression of glucokinase has a limited metabolic impact in islet cells as compared with hepatocytes and suggested that this disparity was related to limited flux through the distal steps of glycolysis in the islet cell (see, e.g., paragraph spanning pages 4551-4552). This hypothesis suggested further research to investigate whether “simultaneous overexpression” of LDH would “be an approach to unveiling the full metabolic impact of glucokinase overexpression, and whether such a maneuver will enhance glucose-stimulated insulin secretion” (p. 4552). This publication does not appreciate that LDH overexpression alone is potentiating for fuel-stimulated IS. Further, the importance of a compartmentalized pool (e.g., mitochondrial) of LDH in enhancing fuel-stimulated IS was not recognized. In representative embodiments of the present invention, an isolated nucleic acid encoding LDH is introduced into a cell, wherein the cell does not comprise an isolated nucleic acid encoding glucokinase and/or does not overexpress glucokinase.

It will be apparent to those skilled in the art that any suitable vector can be used to deliver the isolated nucleic acids of this invention to the target cell(s) or subject of interest. The choice of delivery vector can be made based on a number of factors known in the art, including age and species of the target host, in vitro vs. in vivo delivery, level and persistence of expression desired, intended purpose (e.g., for therapy or drug screening), the target cell or organ, route of delivery, size of the isolated nucleic acid, safety concerns, and the like.

Suitable vectors include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and plant virus satellites.

Protocols for producing recombinant viral vectors and for using viral vectors for nucleic acid delivery can be found in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Particular examples of viral vectors are those previously employed for the delivery of nucleic acids including, for example, retrovirus, adenovirus, AAV, herpes virus, and poxvirus vectors.

In certain embodiments of the present invention, the delivery vector is an adenovirus vector. The term “adenovirus” as used herein is intended to encompass all adenoviruses, including the Mastadenovirus and Aviadenovirus genera. To date, at least forty-seven human serotypes of adenoviruses have been identified (see, e.g., FIELDS et al., VIROLOGY, volume 2, chapter 67 (3d ed., Lippincott-Raven Publishers). Preferably, the adenovirus is a serogroup C adenovirus, still more preferably the adenovirus is serotype 2 (Ad2) or serotype 5 (Ad5).

The various regions of the adenovirus genome have-been mapped and are understood by those skilled in the art (see, e.g., FIELDS et al., VIROLOGY, volume 2, chapters 67 and 68 (3d ed., Lippincoft-Raven Publishers). The genomic sequences of the various Ad serotypes, as well as the nucleotide sequence of the particular coding regions of the Ad genome, are known in the art and can be accessed, e.g., from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

Those skilled in the art will appreciate that the inventive adenovirus vectors can be modified or “targeted” as described in Douglas et al., (1996) Nature Biotechnology 14:1574; U.S. Pat. No. 5,922,315 to Roy et al.; U.S. Pat. No. 5,770,442 to Wickham et al.; and/or U.S. Pat. No. 5,712,136 to Wickham et al.

An adenovirus vector genome or rAd vector genome will typically comprise the Ad terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small heterologous nucleic acid of interest, “stuffer DNA” can be used to maintain the total size of the vector within the desired range by methods known in the art.

Normally, adenoviruses bind to a cell surface receptor (CAR) of susceptible cells via the knob domain of the fiber protein on the virus surface. The fiber knob receptor is a 45 kDa cell surface protein which has potential sites for both glycosylation and phosphorylation. (Bergelson et al., (1997), Science 275:1320-1323). A secondary method of entry for adenovirus is through integrins present on the cell surface. Arginine-Glycine-Aspartic Acid (RGD) sequences of the adenoviral penton base protein bind integrins on the cell surface.

The adenovirus genome can be manipulated such that it encodes and expresses a nucleic acid of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Representative adenoviral vectors derived from the adenovirus strain Ad type 5 d1 324 or other strains of adenovirus (e.g., Ad2, Ad3, Ad7 etc.) are known to those skilled in the art.

Recombinant adenoviruses can be advantageous in certain circumstances in that they are not capable of infecting nondividing cells and can be used to infect a wide variety of cell types, including epithelial cells. Furthermore, the virus particle is relatively stable and amenable to purification and concentration, and can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., as occurs with retroviral DNA). Moreover, the carrying capacity of the adenoviral genome for foreign DNA is large relative to other delivery vectors (Haj-Ahmand and Graham (1986) J. Virol. 57:267).

In particular embodiments, the adenovirus genome contains a deletion therein, so that at least one of the adenovirus genomic regions does not encode a functional protein. For example, first-generation adenovirus vectors are typically deleted for the E1 genes and packaged using a cell that expresses the E1 proteins (e.g., 293 cells). The E3 region is also frequently deleted as well, as there is no need for complementation of this deletion. In addition, deletions in the E4, E2a, protein IX, and fiber protein regions have been described, e.g., by Armentano et al, (1997) J. Virology 71:2408, Gao et al., (1 996) J. Virology 70:8934, Dedieu et al., (1997) J. Virology 71;4626, Wang et al., (1997) Gene Therapy 4:393, U.S. Pat. No. 5,882,877 to Gregory et al. (the disclosures of which are incorporated herein in their entirety). Preferably, the deletions are selected to avoid toxicity to the packaging cell. Wang et al., (1997) Gene Therapy 4:393, has described toxicity from constitutive co-expression of the E4 and E1 genes by a packaging cell line. Toxicity can be avoided by regulating expression of the E1 and/or E4 gene products by an inducible, rather than a constitutive, promoter. Combinations of deletions that avoid toxicity or other deleterious effects on the host cell can be routinely selected by those skilled in the art.

As further examples, in particular embodiments, the adenovirus is deleted in the polymerase (pol), preterminal protein (pTP), IVa2 and/or 100K regions (see, e.g., U.S. Pat. No. 6,328,958; PCT publication WO 00/12740; and PCT publication WO 02/098466; Ding et al., (2002) Mol. Ther. 5:436; Hodges et al., J. Virol. 75:5913; Ding et al., (2001) Hum Gene Ther 12:955; the disclosures of which are incorporated herein by reference in their entireties for the teachings of how to make and use deleted adenovirus vectors for gene delivery).

The term “deleted” adenovirus as used herein refers to the omission of at least one nucleotide from the indicated region of the adenovirus genome. Deletions can be greater than about 1, 2, 3, 5, 10, 20, 50,100, 200, or even 500 nucleotides. Deletions in the various regions of the adenovirus genome can be about at least 1%, 5%, 10%, 25%, 50%, 75%, 90%, 95%, 99%, or more of the indicated region. Alternately, the entire region of the adenovirus genome is deleted. Preferably, the deletion will prevent or essentially prevent the expression of a functional protein from that region. In general, larger deletions are preferred as these have the additional advantage that they will increase the carrying capacity of the deleted adenovirus for a heterologous nucleotide sequence of interest. The various regions of the adenovirus genome have been mapped and are understood by those skilled in the art (see, e.g., FIELDS et al., VIROLOGY, volume 2, chapters 67 and 68 (3d ed., Lippincoft-Raven Publishers).

Those skilled in the art will appreciate that typically, with the exception of the E3 genes, any deletions will need to be complemented in order to propagate (replicate and package) additional virus, e.g., by transcomplementation with a packaging cell.

The present invention can also be practiced with “gutted” adenovirus vectors (as that term is understood in the art, see e.g., Lieber et al., (1996) J. Virol. 70:8944-60) in which essentially all of the adenovirus genomic sequences are deleted.

Adeno-associated viruses (AAV) have also been employed as nucleic acid delivery vectors. For a review, see Muzyczka et al. Curr. Topics in Micro. and Immunol. (1992) 158:97-129). AAV are parvoviruses and have small icosahedral virions, 18-26 nanometers in diameter and contain a single stranded genomic DNA molecule 4-5 kilobases in size. The viruses contain either the sense or antisense strand of the DNA molecule and either strand is incorporated into the virion. Two open reading frames encode a series of Rep and Cap polypeptides. Rep polypeptides (Rep50, Rep52, Rep68 and Rep78) are involved in replication, rescue and integration of the AAV genome, although significant activity can be observed in the absence of all four Rep polypeptides. The Cap proteins (VP1, VP2, VP3) form the virion capsid. Flanking the rep and cap open reading frames at the 5′ and 3′ ends of the genome are 145 basepair inverted terminal repeats (ITRs), the first 125 basepairs of which are capable of forming Y- or T-shaped duplex structures. It has been shown that the ITRs represent the minimal cis sequences required for replication, rescue, packaging and integration of the AAV genome. Typically, in recombinant AAV vectors (rAAV), the entire rep and cap coding regions are excised and replaced with a heterologous nucleic acid of interest.

AAV are among the few viruses that can integrate their DNA into non-dividing cells, and exhibit a high frequency of stable integration into human chromosome 19 (see, for example, Flotte et al. (1992) Am. J. Respir. Cell. Mol. Biol. 7:349-356; Samulski et al., (1989) J Virol. 63:3822-3828; and McLaughlin et.al., (1989) J. Virol. 62:1963-1973). A variety of nucleic acids have been introduced into different cell types using AAV vectors (see, for example, Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

A rAAV vector genome will typically comprise the AAV terminal repeat sequences and packaging signal. An “AAV particle” or “rAAV particle” comprises an AAV vector genome or rAAV vector genome, respectively, packaged within an AAV capsid. The rAAV vector itself need not contain AAV genes encoding the capsid and Rep proteins. In particular embodiments of the invention, the rep and/or cap genes are deleted from the AAV genome. In a representative embodiment, the rAAV vector retains only the terminal AAV sequences (ITRS) necessary for integration, excision, replication.

Sources for the AAV capsid genes can include serotypes AAV-1, AAV-2, AAV-3 (including 3a and 3b), AAV-4, AAV-5, AAV-6, AAV-7, AAV-8, as well as bovine AAV and avian AAV, and any other virus classified by the International Committee on Taxonomy of Viruses (ICTV) as an AAV (see, e.g., BERNARD N. FIELDS et al., VIROLOGY, volume 2, chapter 69 (4th ed., Lippincott-Raven Publishers).

Because of packaging limitations, the total size of the rAAV genome will preferably be less than about 5.2, 5, 4.8, 4.6, 4.5 or 4.2 kb in size.

Any suitable method known in the art can be used to produce AAV vectors expressing the nucleic acids encoding LDH of this invention (see, e.g., U.S. Pat. No. 5,139,941; U.S. Pat. No. 5,858,775; U.S. Pat. No. 6,146,874 for illustrative methods). In one particular method, AAV stocks can be produced by co-transfection of a rep/cap vector encoding AAV packaging functions and the template encoding the AAV vDNA into human cells infected with the helper adenovirus (Samulski et al., (1989) J. Virology 63:3822).

In other particular embodiments, the adenovirus helper virus is a hybrid helper virus that encodes AAV Rep and/or capsid proteins. Hybrid helper Ad/AAV vectors expressing AAV rep and/or cap genes and methods of producing AAV stocks using these reagents are known in the art (see, e.g., U.S. Pat. No. 5,589,377; and U.S. Pat. No. 5,871,982, U.S. Pat. No. 6,251,677; and U.S. Pat. No. 6,387,368). Preferably, the hybrid Ad of the invention expresses the AAV capsid proteins (i.e., VP1, VP2, and VP3). Alternatively, or additionally, the hybrid adenovirus can express one or more of AAV Rep proteins (i.e., Rep40, Rep52, Rep68 and/or Rep78). The AAV sequences can be operatively associated with a tissue-specific or inducible promoter.

The AAV rep and/or cap genes can alternatively be provided by a packaging cell that stably expresses the genes (see, e.g., Gao et al., (1998) Human Gene Therapy 9:2353; Inoue et al., (1998) J. Virol. 72:7024; U.S. Pat. No. 5,837,484; WO 98/27207; U.S. Pat. No. 5,658,785; WO 96/17947).

Another vector for use in the present invention comprises Herpes Simplex Virus (HSV). Herpes simplex virions have an overall diameter of 150 to 200 nm and a genome consisting of one double-stranded DNA molecule that is 120 to 200 kilobases in length. Glycoprotein D (gD) is a structural component of the HSV envelope that mediates virus entry into host cells. The initial interaction of HSV with cell surface heparin sulfate proteoglycans is mediated by another glycoprotein, glycoprotein C (gC) and/or glycoprotein B (gB). This is followed by interaction with one or more of the viral glycoproteins with cellular receptors. It has been shown that glycoprotein D of HSV binds directly to Herpes virus entry mediator (HVEM) of host cells. HVEM is a member of the tumor necrosis factor receptor superfamily (Whitbeck et al., (1997), J. Virol.; 71:6083-6093). Finally, gD, gB and the complex of gH and gL act individually or in combination to trigger pH-independent fusion of the viral envelope with the host cell plasma membrane. The virus itself is transmitted by direct contact and replicates in the skin or mucosal membranes before infecting cells of the nervous system for which HSV has particular tropism. It exhibits both a lytic and a latent function. The lytic cycle results in viral replication and cell death. The latent function allows for the virus to be maintained in the host for an extremely long period of time.

HSV can be modified for the delivery of nucleic acids to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express nucleic acids for a long period of time in the central nervous system as long as the lytic cycle does not occur.

In other particular embodiments of the present invention, the delivery vector of interest is a retrovirus. Retroviruses normally bind to a virus-specific cell surface receptor, e.g., CD4 (for HIV); CAT (for MLV-E; ecotropic Murine leukemic virus E); RAM1/GLVR2 (for murine leukemic virus-A; MLV-A); GLVR1 (for Gibbon Ape leukemia virus (GALV) and Feline leukemia virus B (FeLV-B)). The development of specialized cell lines (termed “packaging cells”) which produce only replication-defective retroviruses has increased the utility of retroviruses for gene therapy, and defective retroviruses are characterized for use in gene transfer for gene therapy purposes (for a review, see Miller, (1990) Blood 76:271). A replication-defective retrovirus can be packaged into virions which can be used to infect a target cell through the use of a helper virus by standard techniques.

Yet another suitable vector is a poxvirus vector. These viruses are very complex, containing more than 100 proteins, although the detailed structure of the virus is presently unknown. Extracellular forms of the virus have two membranes while intracellular particles only have an inner membrane. The outer surface of the virus is made up of lipids and proteins that surround the biconcave core. Poxviruses are antigenically complex, inducing both specific and cross-reacting antibodies after infection. Poxvirus receptors are not presently known, but it is likely that there exists more than one given the tropism of poxvirus for a wide range of cells. Poxvirus gene expression is well studied due to the interest in using vaccinia virus as a vector for expression of nucleic acids.

In addition to viral transfer methods, such as those illustrated above, non-viral methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. Naked plasmids can be introduced into muscle cells by injection into the tissue. Expression can extend over many months, although the number of positive cells is typically low (Wolff et al., (1989) Science 247:247). Cationic lipids have been demonstrated to aid in introduction of nucleic acids into some cells in culture (Felgner and Ringold, (1989) Nature 337:387). Injection of cationic lipid plasmid DNA complexes into the circulation of mice has been shown to result in expression of the DNA in lung (Brigham et al., (1989) Am. J. Med. Sci. 298:278). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., (1992) No Shinkei Geka 20:547; PCT publication WO 91/06309; Japanese patent application 1047381; and European patent publication EP-A-43075).

Liposomes that consist of amphiphilic cationic molecules are useful non-viral vectors for nucleic acid delivery in vitro and in vivo (reviewed in Crystal, Science 270: 404-410 (1995); Blaese et al., Cancer Gene Ther. 2:292-297 (1995); Behr et al., Bioconjugate Chem. 5:382-389 (1994); Remy et al., Bioconjugate Chem. 5:647-654 (1994); and Gao et al., Gene Therapy 2:710-722 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84:7413-17 (1987); Loeffler et al., Methods in Enzymology 217:599-618 (1993); Felgner et al., J. Biol. Chem. 269: 2550-2561 (1994)).

Several groups have reported the use of amphiphilic cationic lipid:nucleic acid complexes for in vivo transfection both in animals and in humans (reviewed in Gao et al., Gene Therapy 2:710-722 (1995); Zhu et al., Science 261:209-211 (1993); and Thierry et al., Proc. Natl. Acad. Sci. USA 92:9742-9746 (1995)). U.S. Pat. No. 6,410,049 describes a method of preparing cationic lipid:nucleic acid complexes that have a prolonged shelf life.

VI. Subjects, Pharmaceutical Formulations, Dosages and Modes of Administration.

The present invention finds use in veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, humans, non-human primates, bovines, ovines, caprines, equines, felines, canines, lagomorphs, rats, mice, etc. In particular embodiments, the subject is a human subject that has been diagnosed with diabetes mellitus (non-insulin dependent or insulin dependent), is obese or otherwise has impaired glucose tolerance. Human subjects include neonates, infants, juveniles, and adults. In other embodiments, the subject is an animal model of diabetes, obesity or impaired glucose tolerance. In other particular embodiments, the subject is a subject in need of the therapeutic methods of the invention, e.g., because the subject is diagnosed with a glucose intolerant conditions such as diabetes, is suspected of having such a condition, or is at risk of developing such a condition.

As one particular aspect, the invention provides a pharmaceutical formulation comprising a compound that modulates LDH activity and/or IS activity in a pharmaceutically acceptable carrier. As another aspect, the present invention provides a pharmaceutical formulation comprising a compound identified according to the screening methods of this invention in a pharmaceutically acceptable carrier.

In other particular embodiments, the present invention provides a pharmaceutical formulations comprising an isolated nucleic acid or vector of the invention in a pharmaceutically-acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The compounds of the invention can be formulated for administration in a pharmaceutical carrier in accordance with known techniques. See, e.g., Remington, The Science And Practice of Pharmacy (9th Ed. 1995). In the manufacture of a pharmaceutical formulation according to the invention, the compound (including the physiologically acceptable salts thereof) is typically admixed with, inter alia, an acceptable carrier. The carrier can be a solid or a liquid, or both, and is preferably formulated with the compound as a unit-dose formulation, for example, a tablet, which can contain from 0.01 or 0.5% to 95% or 99% by weight of the compound. One or more compounds can be incorporated in the formulations of the invention, which can be prepared by any of the well-known techniques of pharmacy.

A further aspect of the invention is a method of treating subjects in vivo with the inventive compounds of the invention, comprising administering to a subject a pharmaceutical composition comprising a compound of the invention in a pharmaceutically acceptable carrier, wherein the pharmaceutical composition is administered in a therapeutically effective amount. Administration of the compounds of the present invention to a human subject or an animal in need thereof can be by any means known in the art for administering compounds.

The formulations of the invention include those suitable for oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal and inhalation administration, administration to the liver by intraportal delivery, as well as direct organ injection (e.g., into the liver, into the brain for delivery to the central nervous system, into the pancreas). The most suitable route in any given case will depend on the nature and severity of the condition being treated and on the nature of the particular compound which is being used.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution of the compound. Suitable formulations comprise citrate or bis/tris buffer (pH 6) or ethanol/water and contain from 0.1 to 0.2M of the compound.

The compound can alternatively be formulated for nasal administration or otherwise administered to the lungs of a subject by any suitable means, but is preferably administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159. Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g., Remington's Pharmaceutical Sciences (Maack Publishing Co., Easton, Pa. The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly about 22 μmol/kg and 33 μmol/kg of the compound for intravenous or oral administration.

A further aspect of the invention is a method of treating subjects in vivo with the isolated nucleic acids and delivery vectors of this invention. Administration of the delivery vectors of the present invention to a human subject or an animal can be by any means known in the art for administering vectors. The subject can be an obese subject, a diabetic subject (non-insulin dependent or insulin dependent diabetes) or otherwise have impaired glucose tolerance.

In one representative embodiment, the invention provides a method of enhancing fuel-stimulated IS (e.g., glucose-stimulated IS) in a subject comprising, administering to the subject an isolated nucleic acid encoding LDH and/or a compound of the invention in an amount effective to enhance fuel-stimulated insulin secretion (e.g., in response to an elevation of serum concentrations of secretagogues such as glucose, glyceraldehyde, glycerol, amino acids and/or fatty acids). Alternatively stated, the isolated nucleic acid encoding LDH and/or compound is administered in an amount effective to result in an improvement in glucose tolerance in the subject. The isolated nucleic acid can be provided in a delivery vector, as described above.

In particular embodiments, an isolated nucleic acid encoding the monocarboxylate transporter is also delivered to the subject (see, e.g., Ishihara et al., (1999) J. Clin. Invest. 104:1621). Co-expression of a nucleic acid encoding recombinant LDH and a nucleic acid encoding monocarboxylate transporter can result in higher levels of IS because of elevated intracellular lactate concentrations.

Isolated nucleic acids and vectors can be formulated as described above for compounds of the invention.

Dosages will depend upon the mode of administration, the severity of the disease or condition to be treated, the individual subject's condition, age and species of the subject, the particular vector, and the nucleic acid to be delivered, and can be determined in a routine manner. In particular embodiments, the vector is administered to the subject in a therapeutically effective amount, as that term is defined above.

Typically, with respect to viral vectors, at least about 10³ virus particles, at least about 10⁵ virus particles, at least about 10⁷ virus particles, at least about 10⁹ virus particles, at least about 10¹¹ virus particles, at least about 10¹² virus particles, or at least about 10¹³ virus particles are administered to the subject per treatment. Exemplary doses are virus titers of about 10⁷ to about 10¹⁵ particles, about 10⁷to about 10¹⁴ particles, about 10⁸to about 10¹³ particles, about 10¹⁰ to about 10¹⁵ particles, about 10¹¹ to about 10¹⁵ particles, about 10¹² to about 10¹⁴ particles, or about 10¹² to about 10¹³ particles.

In particular embodiments of the invention, more than one administration (e.g., two, three, four, or more administrations) can be employed over a variety of time intervals (e.g., hourly, daily, weekly, monthly, etc.) to achieve therapeutic levels of nucleic acid expression.

VII. Expression Systems for Recombinant LDH.

As indicated above, LDH polypeptide can be produced in, and optionally purified from, cultured cells or organisms expressing a nucleic acid encoding LDH for a variety of purposes (e.g., screening assays, large-scale protein production, therapeutic methods based on delivery of purified LDH or insulin).

In particular embodiments, an isolated nucleic acid encoding LDH can be introduced into a cultured cell, e.g., a cell of a primary or immortalized cell line for recombinant protein production. The recombinant cells can be used to produce the LDH polypeptide, which is collected from the cells or cell culture medium. Likewise, recombinant protein can be produced in, and optionally purified from an organism (e.g., a microorganism, animal or plant) being used essentially as a bioreactor.

Generally, the isolated nucleic acid is incorporated into an expression vector (viral or nonviral as described above). Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding an LDH subunit operatively associated with the promoter, and, optionally, a termination sequence including a stop signal for RNA polymerase and a polyadenylation signal for polyadenylase.

Expression vectors can be designed for expression of polypeptides in prokaryotic or eukaryotic cells. For example, polypeptides can be expressed in bacterial cells such as E. coli, insect cells (e.g., in the baculovirus expression system), yeast cells or mammalian cells. Some suitable host cells are discussed further in Goeddel, Gene Expression Technology: Methods in Enzymology 185, Academic Press, San Diego, Calif. (1990). Examples of vectors for expression in yeast S. cerevisiae include pYepSecl (Baldari et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of nucleic acids to produce proteins in cultured insect cells (e.g., Sf 9 cells) include the pAc series (Smith et al., (1983) Mol. Cell. Biol. 3:2156-2165) and the pVL series (Lucklow, V. A., and Summers, M. d. (1989) Virology 170:31-39).

Examples of mammalian expression vectors include pCDM8 (Seed, (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, adenovirus 2, cytomegalovirus and Simian Virus 40.

In addition to the regulatory control sequences discussed above, the recombinant expression vector can contain additional nucleotide sequences. For example, the recombinant expression vector can encode a selectable marker gene to identify host cells that have incorporated the vector.

Vectors can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

Often only a small fraction of cells (in particular, mammalian cells) integrate the foreign DNA into their genome. In order to identify and select these integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

Recombinant proteins can also be produced in a transgenic plant in which the isolated nucleic acid encoding the protein is inserted into the nuclear or plastidic genome. Plant transformation is known as the art. See, in general, Methods in Enzymology Vol. 153 (“Recombinant DNA Part D”) 1987, Wu and Grossman Eds., Academic Press and European Patent Application EP 693554.

Foreign nucleic acids can be introduced into plant cells or protoplasts by several methods. For example, nucleic acid can be mechanically transferred by microinjection directly into plant cells by use of micropipettes. Foreign nucleic acid can also be transferred into a plant cell by using polyethylene glycol which forms a precipitation complex with the genetic material that is taken up by the cell (Paszkowski et al. (1984) EMBO J. 3:2712-22). Foreign nucleic acid can be introduced into a plant cell by electroporation (Fromm et al. (1985) Proc. Natl. Acad. Sci. USA 82:5824). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells comprising the foreign nucleic acid can be accomplished using phenotypic markers.

Cauliflower mosaic virus (CaMV) can be used as a vector for introducing foreign nucleic acids into plant cells (Hohn et al. (1982) “Molecular Biology of Plant Tumors,” Academic Press, New York, pp. 549-560; Howell, U.S. Pat. No. 4,407,956). CaMV viral DNA genome is inserted into a parent bacterial plasmid creating a recombinant DNA molecule which can be propagated in bacteria. The recombinant plasmid can be further modified by introduction of the desired DNA sequence. The modified viral portion of the recombinant plasmid is then excised from the parent bacterial plasmid, and used to inoculate the plant cells or plants.

High velocity ballistic penetration by small particles can be used to introduce foreign nucleic acid into plant cells. Nucleic acid is disposed within the matrix of small beads or particles, or on the surface (Klein et al. (1987) Nature 327:70-73). Although typically only a single introduction of a new nucleic acid segment is required, this method also provides for multiple introductions.

A nucleic acid can be introduced into a plant cell by infection of a plant cell, an explant, a meristem or a seed with Agrobacterium tumefaciens transformed with the nucleic acid. Under appropriate conditions, the transformed plant cells are grown to form shoots, roots, and develop further into plants. The nucleic acids can be introduced into plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al. (1984) “Inheritance of Functional Foreign Genes in Plants,” Science 233:496498; Fraley et al. (1983) Proc. Natl: Acad. Sci. USA 80:4803).

Plants from which protoplasts can be isolated and cultured to give whole regenerated plants can be transformed so that whole plants are recovered which contain the transferred foreign nucleic acid. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Ciohorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Plant regeneration from cultured protoplasts is described in Evans et al., “Protoplasts Isolation and Culture,” Handbook of Plant Cell Cultures 1:124-176 (MacMillan Publishing Co. New York 1983); M. R. Davey, “Recent Developments in the Culture and Regeneration of Plant Protoplasts,” Protoplasts (1983)-Lecture Proceedings, pp. 12-29, (Birkhauser, Basal 1983); P. J. Dale, “Protoplast Culture and Plant Regeneration of Cereals and Other Recalcitrant Crops,” Protoplasts (1983)-Lecture Proceedings, pp. 31-41, (Birkhauser, Basel 1983); and H. Binding, “Regeneration of Plants,” Plant Protoplasts, pp. 21-73, (CRC Press, Boca Raton 1985).

Regeneration from protoplasts varies from species to species of plants, but generally a suspension of transformed protoplasts containing copies of the exogenous sequence is first generated. In certain species, embryo formation can then be induced from the protoplast suspension, to the stage of ripening and germination as natural embryos. The culture media can contain various amino acids and hormones, such as auxin and cytokinins. It can also be advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

Methods for generation of genetically engineered plants are further described in U.S. Pat. Nos. 5,283,184, 5,482,852, and European Patent Application EP 693 554, all of which are hereby incorporated by reference.

Having now described the invention, the same will be illustrated with reference to certain examples, which are included herein for illustration purposes only, and which are not intended to be limiting of the invention.

### EXAMPLE 1

### Cell Culture

The cell line 832/13 was created from INS-1 insulinoma β-cells as described earlier (Hohmeier et al. (2000) Diabetes 49:424430). Cells were cultured in RPMI-1640 medium containing 11 mM glucose supplemented with 10% fetal bovine serum, 10 mM HEPES, 2 mM glutamine, 1 mM sodium pyruvate and 50 mM β-mercaptoethanol. Media was routinely changed every second day.

### EXAMPLE 2

### Recombinant Adenovirus

A cDNA clone containing the coding sequence of the rat cytoplasmic LDH_{A} isoform surrounded by a EcoRI/BamHI linker (underlined in primers) was made by PCR amplification of rat liver cDNA using the following primers 5′-G\overline{GA} \overline{ATT} \overline{C}GT GTG CTG GAG CCA CTG T-3′ (sense, SEQ ID NO:12), and 5′-CGC \overline{GGA} \overline{TCC} TGT AGA ACA TTT TAT GCA C-3′ (antisense, SEQ ID NO:13), and subsequently ligated into the EcoRI/BamHI sites of pAC CMV.pLpA. After sequencing, pAcCMV.pLpA/LDH_{A} was cotransfected with pJM17 into low passage HK-293 cells for virus propagation as described earlier (Becker et al. (1994) Methods Cell Biol. 43(Pt A):161-89; Becker et al., (1994) J. Biol. Chem.269:21234-8).

### EXAMPLE 3

### Cloning of Mitochondrial Form of LDH_{A}

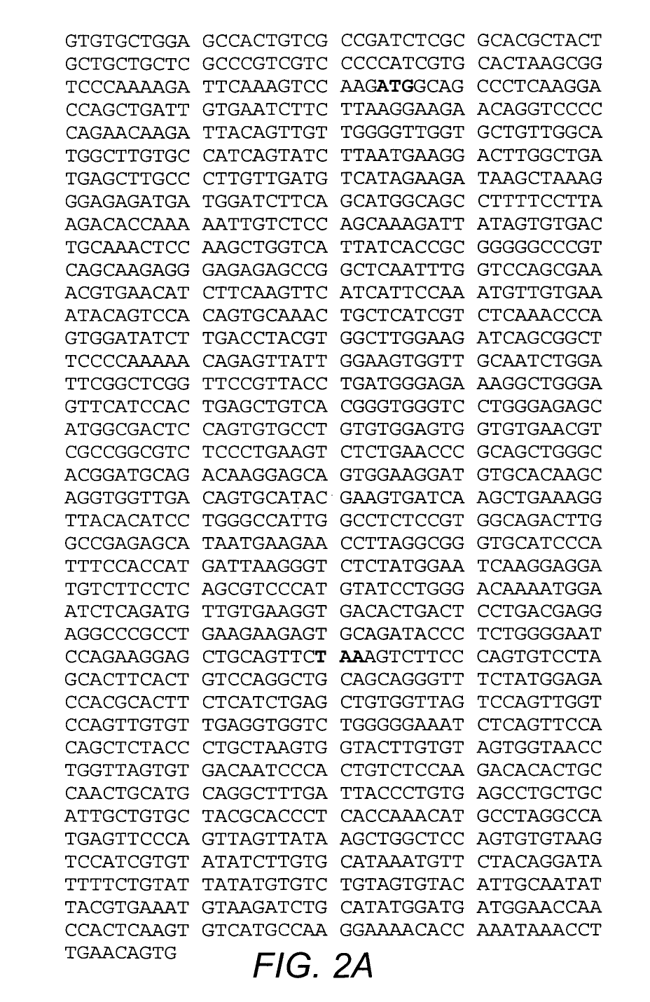

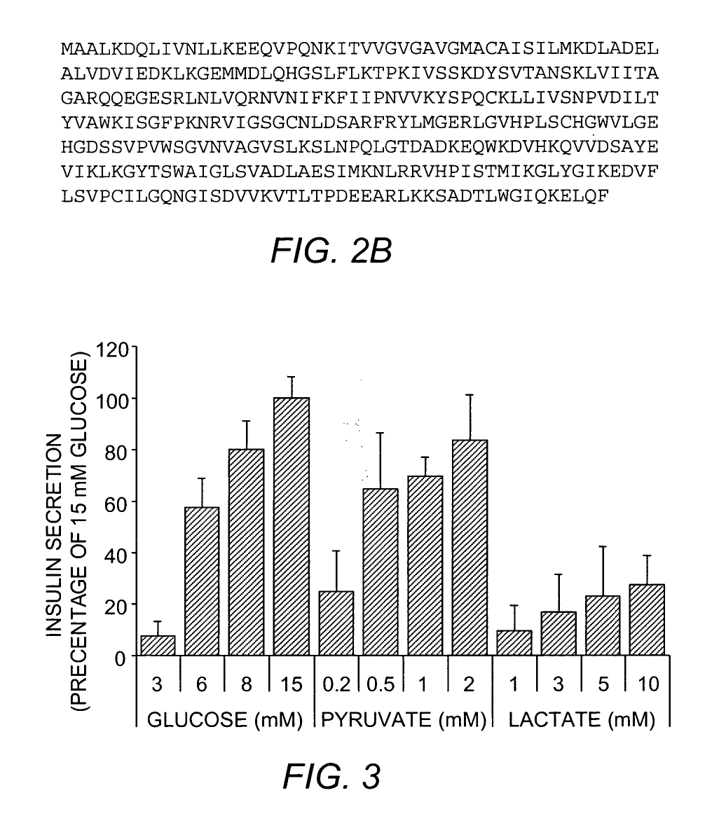

DNase treated RNA purified from rat liver by RNeasy kit (Qiagen) was utilized as template for cloning of the rat mitochondrial form of LDH_{A} using SuperScript one-step RT-PCR with platinum Taq (Invitrogen) and the following primers 5′-CGC TCT ACT TGC TGT AGG-3′ (sense 5′LDHexonmit, SEQ ID NO:14) and 5′-GCC TGG ACA GTG AAG TGC TAG G-3′ (antisense 3′cloning SEQ ID NO:15). RT-PCR reaction was performed according to the manufacturer's recommendations with the following cycling conditions: cDNA synthesis and pre-denaturation: 50° C. for 30 min, 94° C. for 2 min. PCR amplification: 2 cycles (94° C. for 15 s, 56° C. for 30 s, 68° C. for 90 s), 2 cycles (94° C. for 15 s, 54° C. for 30 s, 68° C. for 90 s), 30 cycles (94° C. for 15 s, 52° C. for 30 s, 68° C. for 90 s). Final extension 72° C. for 5 min. 1 μl PCR reaction was then used as template for an additional PCR reaction with the following cycling conditions 94° C. for 2 min, 30 cycles (94° C. for 15 s, 53° C. for 30 s, 72° C. for 90 s) and 72° C. for 5 min. The resulting PCR product of 1.2 kb was cloned into the TOPO® TA blunt vector (Invitrogen) and sequenced (FIG. 2A; SEQ ID NO:3).

### EXAMPLE 4

### Recombinant MitLDH_{A} Adenovirus

With the purpose of constructing a mitLDH_{A} adenovirus, DNAse treated RNA purified from rat liver by RNeasy kit (Qiagen) was utilized as template for cloning of the mitochondrial form of LDH using SuperScript one-step RT-PCR with platinum Taq (Invitrogen) and the following primers 5′-AAC CGT GTA AGA GGA GGG ACC ATC-3′ (sense, SEQ ID NO:16) and 5′-TGG ACC AAC TGG ACT AAC CAC AGC-3′ (antisense, SEQ ID NO:17). Cycling conditions: cDNA synthesis and pre-denaturation: 53° C. for 30 min, 94° C. for 2 min. PCR amplification: 2 cycles (94° C. for 15 s, 63° C. for 30 s, 68° C. for 90 s), 2 cycles (94° C. for 15 s, 61° C. for 30 s, 68° C. for 90 s), 30 cycles (94° C. for 15 s, 59° C. for 30 s, 68° C. for 90 s). Final extension 72° C. for 5 min. The resulting PCR product of 1.2 kb was cloned into the TOPO® TA blunt vector (Invitrogen). Subsequently, the insert was cut out with EcoRI and ligated into pAC CMV.pLpA and resulting clones were tested for orientation by restrictions digest and sequencing. Virus were made as described above.

### EXAMPLE 5

### Virus Transduction

832/13 cells were split to a cell density of 20-30 % confluence in 12 well plates. At a cell density of 90%, cells were exposed to virus by adding crude extract directly to the growth media. After 16 hours of transduction, virus containing media was removed and the cells were cultured in fresh media in additional 24 hours before secretion assays.

### EXAMPLE 6

### Secretion Assays

Insulin secretion was assayed in HEPES buffered saline solution (HBSS) (114 mM NaCl, 4.7 mM KCl, 1.2 mM KH₂PO₄, 1.16 mM MgSO₄, 20 mM HEPES, 2.5 mM CaCl₂, 25.5 mM NaHCO₃, pH 7.2 containing 0.2% bovine serum albumin (essentially fatty acid free). Cells were washed in 1 ml of HBSS, preincubated in 1.5 ml of HBSS for 2 hour prior to a 2 hour incubation in the presence of secretagogues as indicated in the figures. The insulin levels were determined by radioimmunoassay with the Coat-A-Count kit (ICN Pharmaceuticals, Costa Mesa, Calif.) and lactate output was determined with a Lactate Reagent kit (Sigma, St. Louis, Mo.) according to protocols outlined by the manufacturer.

### EXAMPLE 7

### LDH Activity Assay and Protein Levels

832/13 cells in 12 well plates were washed in 1 ml Phosphate Buffered Saline (PBS, 10 mM Potassium phosphate, 120 mM NaCl, 2.7 mM KCl, pH 7.4) and subsequently lysed in 50 ml PBS containing 0.1% Triton X100. For LDH activity, 0.5-10 ml of extract was incubated in a buffer containing 50 mM potassium phosphate, 0.63 mM sodium pyruvate, pH 7.5, and the enzyme reaction is initiated by adding NADH to a final concentration of 0.18 mM, and activity was measured by the decrease in absorbance at 340 nm at RT. Protein levels were determined using a Bio-Rad protein assay (Bio-Rad, Hercules, Calif.) according to protocols outlined by the manufacturer.

### EXAMPLE 8

### Glucose Usage

In order to determine glycolytic flux, the insulin secretion assay was performed in the presence [³H] glucose (specific activity 2×10⁴ cpm/ml). After 2 hours incubation, the HBSS was collected, proteins were removed by centrifugation (2 min, 12000 g) after adding trichloric acid to a final concentration of 4%. Microtubes (1.5 ml without cap) containing the supernatants were placed in tightly closed scintillation tubes containing 500 μl H₂O and placed at 50° C. overnight. After cooling to RT, glucose usage was determined from the level of ³H₂O released to the scintillation tubes.

### EXAMPLE 9

### Effect of LDH_{A} on Glucose and Pyruvate Stimulation of IS

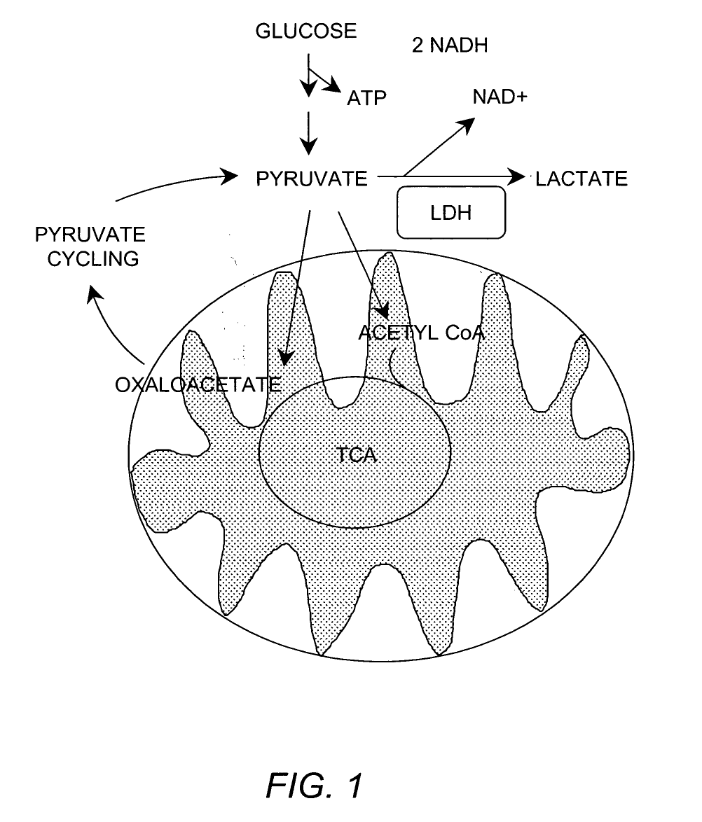

LDH catalyzes the conversion of pyruvate to lactate (FIG. 1). Based on earlier observations showing a direct correlation between pyruvate cycling and insulin secretion, it was hypothesized that the effect of overexpression of LDH_{A}—if any—would be impaired IS response consistent with earlier observations (Alcazar et al. (2000) Biochem. J. 352:373-380; Ishihara et al. (1999) J. Clin. Invest. 104:1621-1629; Ainscow et al. (2000) Diabetes 49:1149-1155). Lactate has been observed to be a poor secretagogue compared to pyruvate and glucose of insulin (FIG. 3).

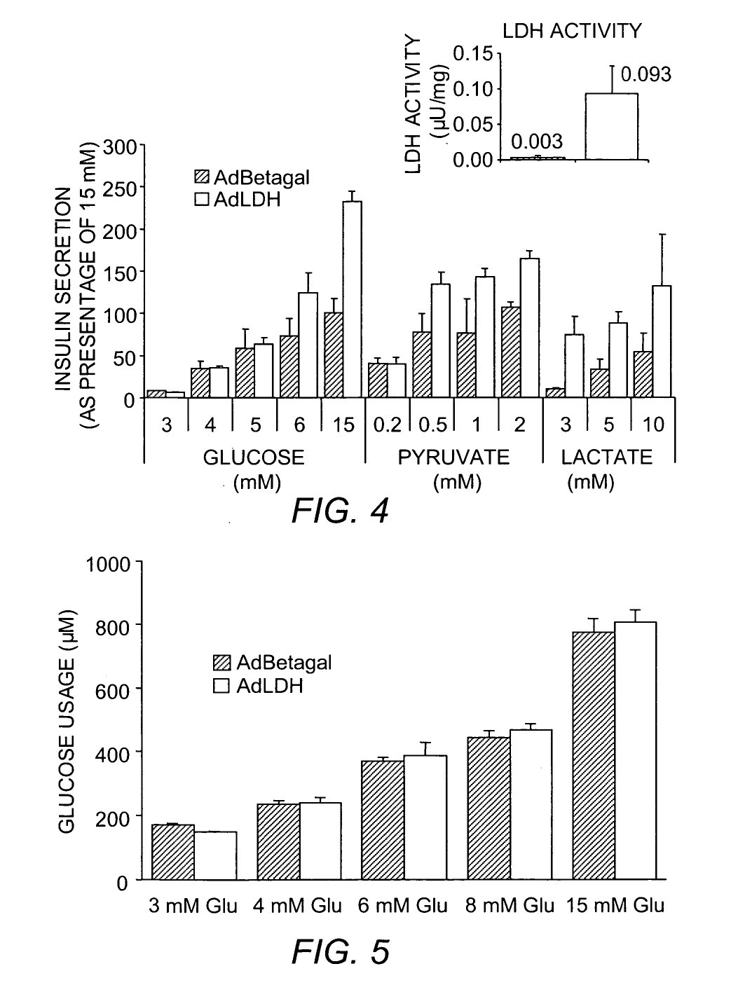

Surprisingly, overexpression of rat LDH_{A} in our 832/13 cell model potentiates IS in response to glucose and pyruvate (FIG. 4) without affecting glycolytic flux with glucose as secretagogue (FIG. 5). In the absence of the overexpression of LDH_{A}, lactate is a weak stimulator of IS. However, in 832/13 cells treated with adenovirus expressing rat LDH_{A} (AdLDH), lactate stimulation of IS was potentiated (FIG. 4). In cells overexpressing LDH_{A}, lactate is as effective as glucose or pyruvate as an insulin secretagogue.

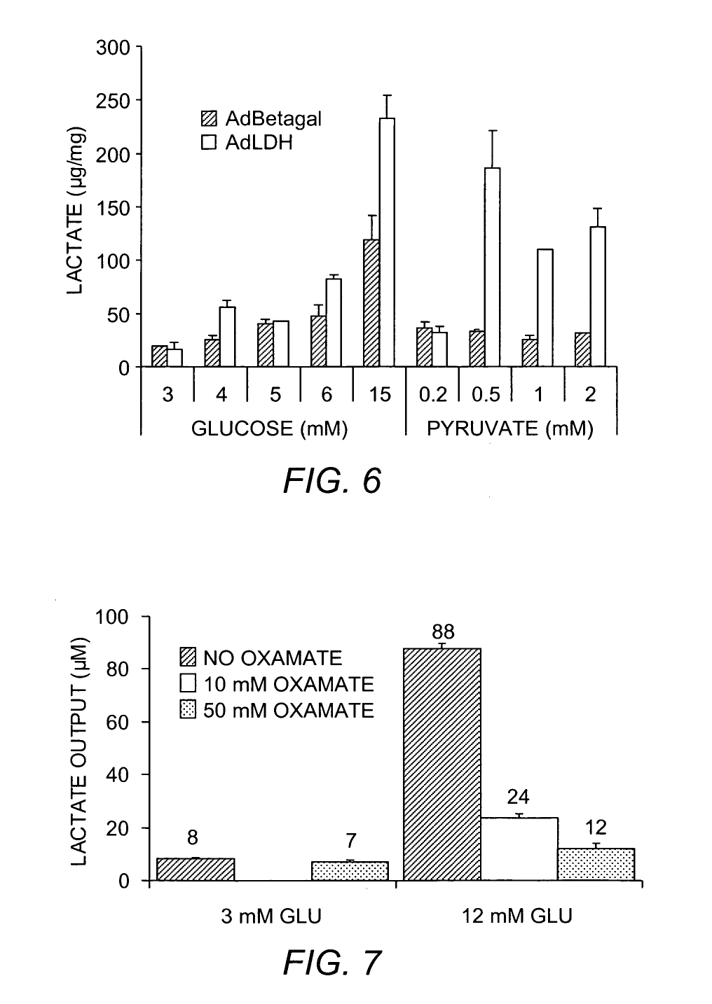

When lactate production was examined, it was observed that rat LDH_{A} overexpression increases lactate output in response to both glucose and pyruvate (FIG. 6), showing that the LDH driven process occurs in the direction of lactate. This indicates that lactate itself might be involved in regulation of IS. However, in the absence of LDH_{A} overexpression, lactate is only a weak stimulator of IS (FIG. 3).

### EXAMPLE 10

### Effect of Oxamate on Pyruvate Cycling and IS

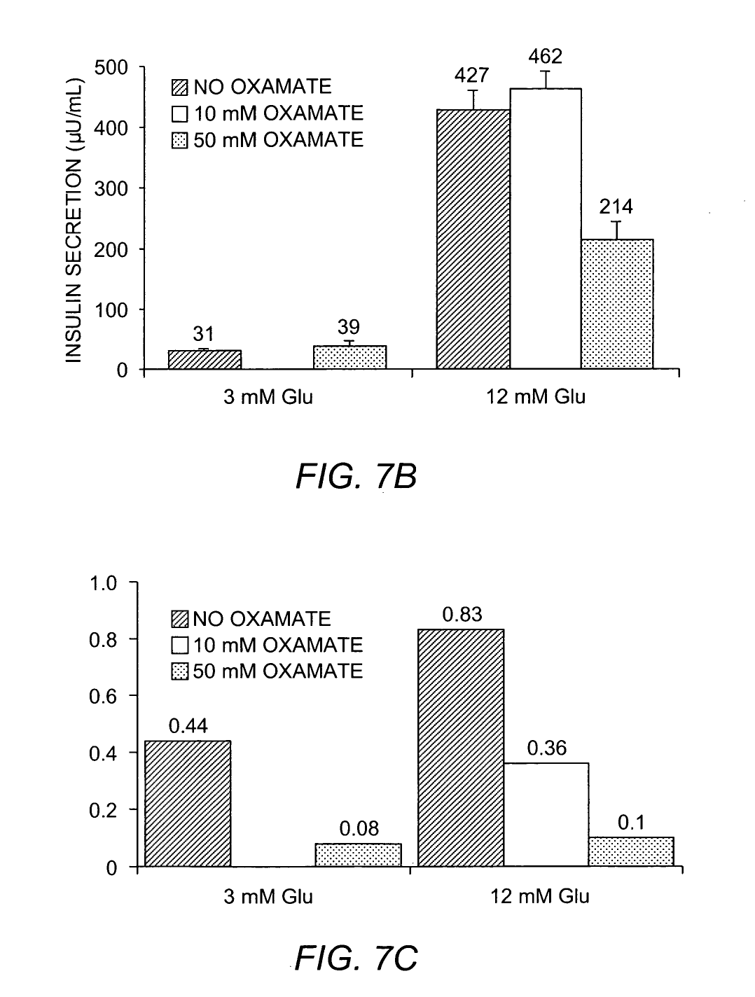

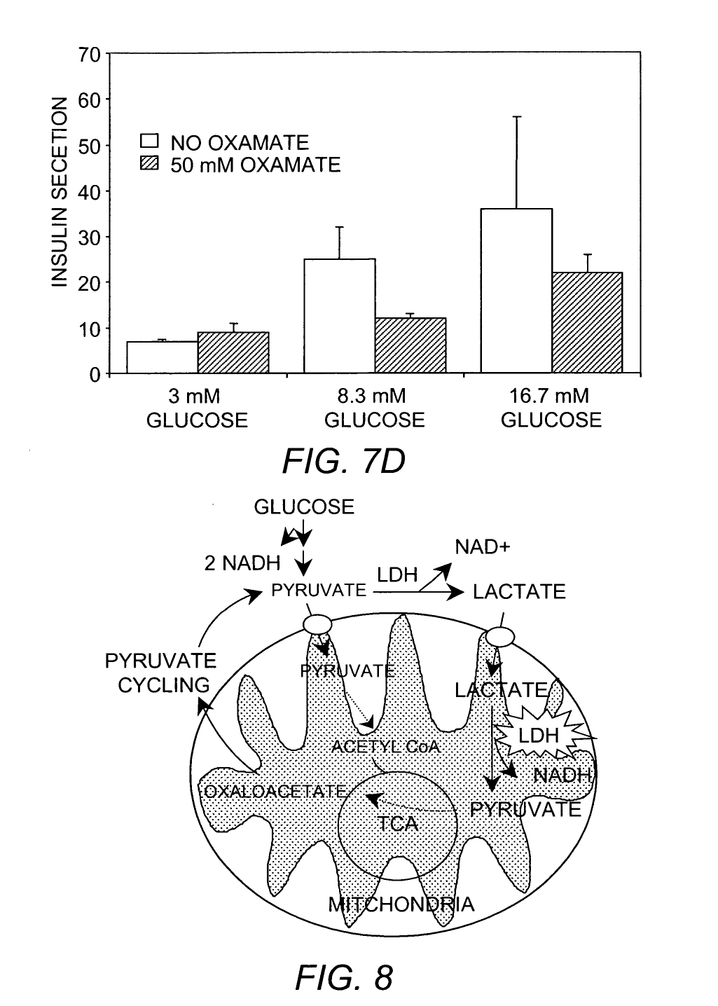

¹³C NMR analyses demonstrated a correlation between lactate output, pyruvate cycling and insulin secretion in the presence and absence of the LDH inhibitor, oxamate in 823/13 cells (FIGS. 7A-C). Oxamate inhibition of glucose-stimulated insulin secretion was also observed in pancreatic islet β-cells (FIG. 7D).

### EXAMPLE 11

### Mitochondrial LDH_{A} is Implicated in Fuel-Stimulated IS

The results shown in Example 10 are consistent with the regulation of fuel-stimulated IS involving a mitochondrial form of LDH following the scheme shown in FIG. 8. According to this model, both lactate and pyruvate are transported into the mitochondria via the monocarboxylate transporter. Inside the mitochondria, lactate is then converted to pyruvate via the mitochondrial LDH. In our model, both entry pathways must be active in order to obtain IS. With lactate as secretagogue, the flux though the pyruvate entry path is limiting due to low level of LDH. However, when LDH is overexpressed, the flux though the pyruvate entry pathway increases which in turn increases IS. On the other hand, with pyruvate or glucose as secretagogues, the lactate entry way is limiting for IS and an increase in LDH expression will increase the flux thereby resulting in a potentiation of IS. In addition, this scheme can also explain our earlier observation of two distinct pools of pyruvate present in beta cells (Lu et al., (2000) Proc. Natl. Acad. Sci. USA 99:2708).

### EXAMPLE 12

### Correlation of Pyruvate Cycling With Lactate Production in Glycerol Kinase Producing Cells

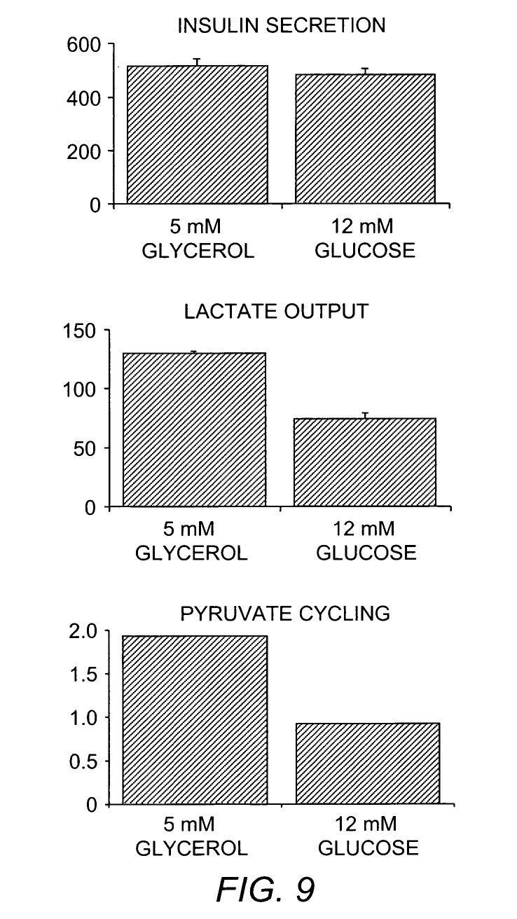

There is minimal expression-of glycerol kinase in β-cells, thus glycerol is not converted to glucose in β-cells, and does not stimulate insulin secretion. FIG. 9 shows ¹³C studies that indicate lactate output correlates with pyruvate cycling in glycerol kinase overexpressing 832/13 cells. Glucose and glycerol stimulate insulin secretion to the same extent in glycerol kinase overexpressing cells. In contrast, pyruvate cycling and lactate output are higher in response to glycerol than to glucose.

### EXAMPLE 13

### Organization of the LDH_{A} Gene and the Identification of an Alternative Exon

The structure of the mouse LDH_{A} gene (GenBank Accession No. Y00309) is shown in FIG. 10. The gene spans 12.9 kb and the cytosolic LDH_{A} mRNA contains 8 exons. Analysis of the complete mouse LDH_{A} gene sequence revealed an alternative exon (SEQ ID NO:18), when spliced to the 5′ end of exon 2 through alternative exon usage will give rise to a mRNA containing an amino-terminal extension of the LDH protein sequence of 29 amino acids (FIG. 7, SEQ ID NO:20). FIG. 11 depicts the leader sequences for mouse (SEQ ID NO:20), rat (SEQ ID NO:21), and human (SEQ ID NO:22) mit-LDH_{A}. Conservation of the amino acid sequence of this 29 amino acid leader peptide sequence was revealed and a consensus sequence (SEQ ID NO:23) from these sequences is shown (FIG. 11).

### EXAMPLE 14

### Cloning of the Mitochondrial Form of LDH_{A} From Rat

Utilizing the PCR primers and protocol outlined in Example 3, a cDNA clone containing the mitochondrial form of rat LDH_{A} (mitLDH_{A}) was isolated. The sequence of this cDNA clone (SEQ ID NO:3) is shown in FIG. 12A. The translation of the open reading frame from nucleotides 101-1186 giving rise to a protein of 361 amino acids (FIG. 12B, SEQ ID NO:4) having a 29 amino acid leader sequence added to the cytoplasmic LDH_{A} polypeptide sequence (FIG. 2B, SEQ ID NO:2). The presence of this transcript in pancreatic 832/13 cells is confirmed by RT-PCR.

### EXAMPLE 15

### Comparisons of Mitochondrial Forms of LDH_{A} From Rat, Mouse, and Human

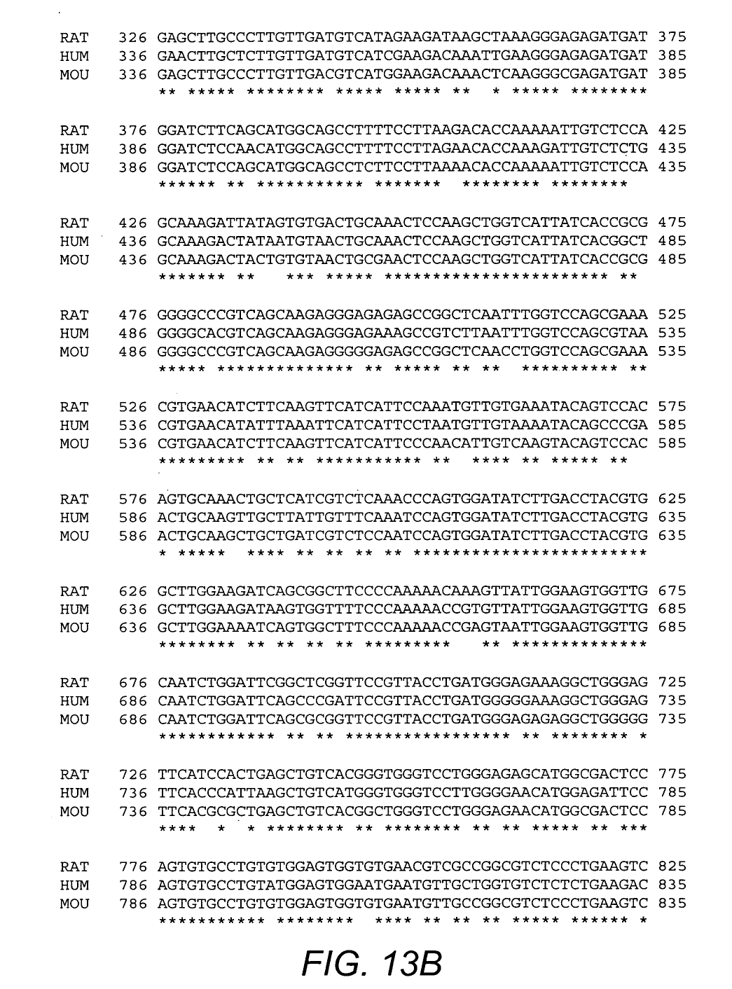

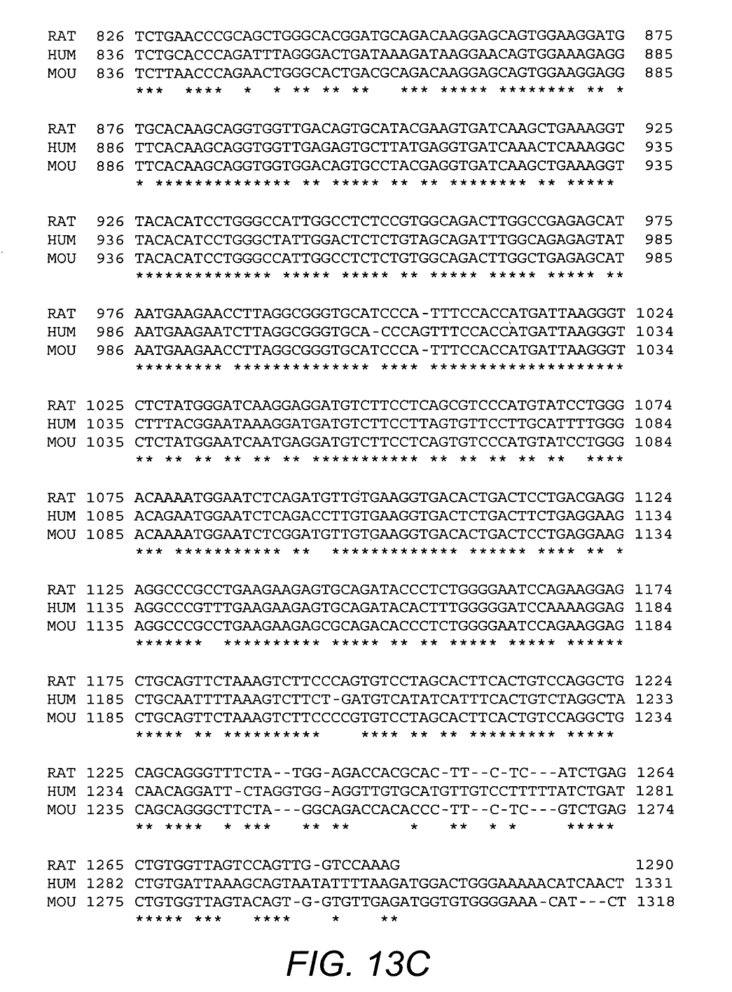

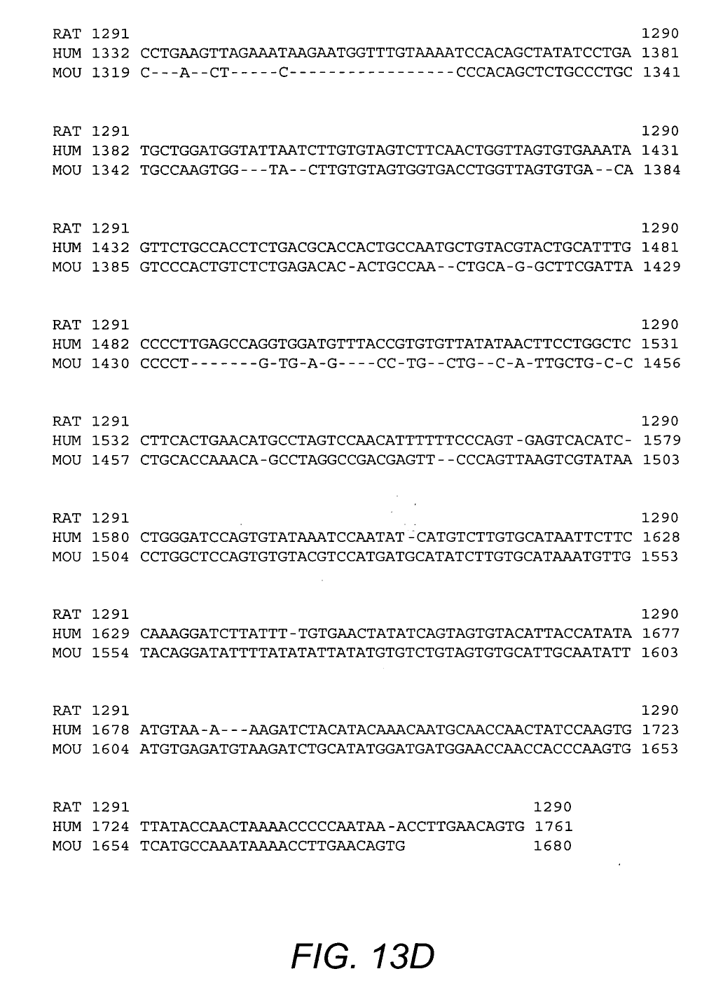

The mouse mRNA (SEQ ID NO:24), the translation of the open reading frame from nucleotides 111-1193 for the mouse mitochondrial LDH (SEQ ID NO:25) are shown in FIGS. 12C and 12D respectively. The human mRNA (SEQ ID NO:26), the translation of the open reading frame from nucleotides 111-1193 for the human mitochondrial LDH (SEQ ID NO:27) are shown in FIGS. 12E and 12F respectively. An alignment of the mouse and human mRNAs with the sequence from the rat cDNA (SEQ ID NO:3) for mitochondrial LDH is shown in FIGS. 13 A-D.

### EXAMPLE 16

### Effect of the Mitochondrial Form of LDH_{A} on Fuel Mediated IS

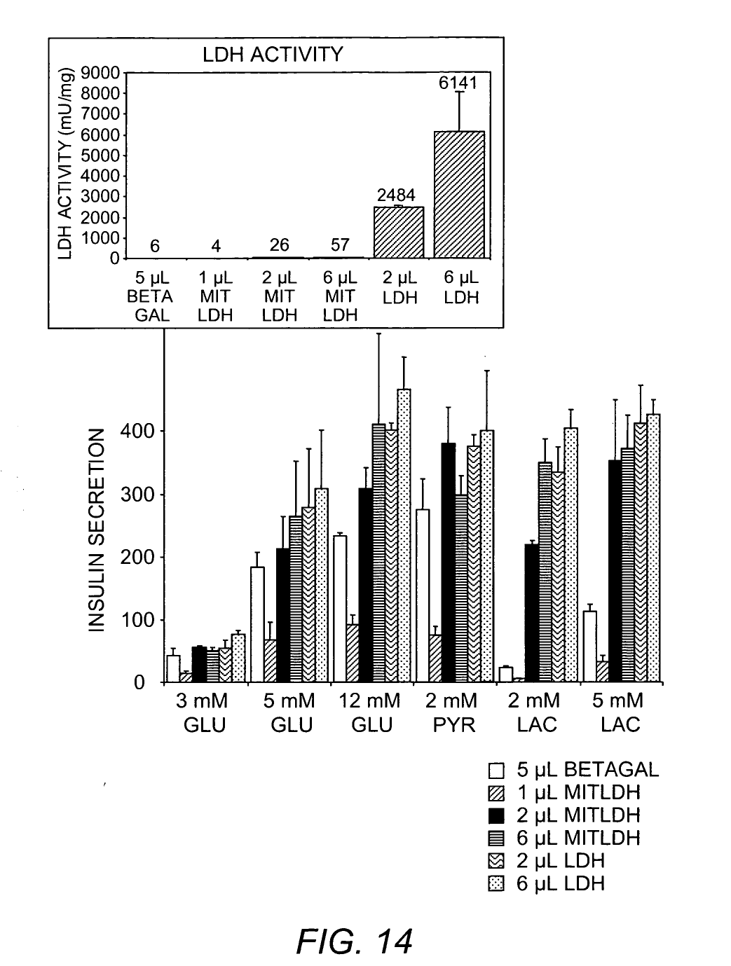

Example 9 describes the effects of overexpression of LDH_{A} on glucose, pyruvate, and lactate mediated IS. The effects of rat mitLDH_{A} on fuel mediated IS were examined and compared with rat cytosolic LDH_{A}, the results of which are shown in FIG. 14. These results show that mitLDH_{A} is capable of potentiating fuel-mediated IS in a similar manner to rat LDH_{A}, and that the magnitude of the mit-LDH_{A} response occurs at lower enzyme activity this is observed with rat LDH_{A}.

### EXAMPLE 17

### Mass Spectroscopy Based Metabolic Profiling

A mass-spectroscopy (MS) based metabolic profiling approach has been used to understand key differences between robustly glucose responsive and poorly glucose responsive INS-1-derived cell lines. These investigations have involved analysis of glucose-derived metabolites via gas chromatography (GC)/MS. In a comparison of a robustly glucose responsive cell line 832/13 with a poorly glucose responsive cell line 832/2, new evidence has been found for a role of lactate and pyruvate in glucose stimulated insulin secretion. The analysis involved stimulation of 832/13 or 832/2 cells with 12 mM glucose for 15 minutes, followed by collection of the cellular media and preparation of cell lysates. Focusing on lactate and pyruvate (other intermediates also show interesting changes, and are the subject of ongoing investigations), Table 2 shows that the ratio of lacate_{lysate}: lactate_{media} and pyruvate_{lysate}: pyruvate_{media} is approximately 10-fold higher in 832/13 cells than in 832/2 cells. In other words, the more glucose responsive cell line retains a much higher percentage of lactate and pyruvate within the cell than the less glucose responsive line. One possible explanation for this result is lesser activity of monocarboxylic acid transporters in the former cells than in the latter. This finding suggests that inhibition of monocarboxylic acid transport can enhance glucose stimulated insulin secretion in 832/2 cells and other cells that lack a robust glucose stimulated insulin secretory response.

TABLE 2
Lactate and pyruvate ratios in cell lysates versus media following
15 minutes of stimulation with 12 mM glucose.

| | Lacate_{lysate}:Lactate_{media} | Pyruvate_{lysate}:Pyruvate_{media} |
|---|---|---|
| 832/13 Cells | 75 | 10 |
| 832/2 Cells | 8.5 | 1.2 |

### EXAMPLE 18

### RNAi Inhibition of Cytosolic LDH Activity

As shown in the previous Examples, the LDH inhibitor oxamate potently inhibits glucose stimulated insulin secretion from 832/13 cells and normal rat islets. Investigations are carried out to provide independent evidence that glucose stimulated insulin secretion is impaired by inhibition of the cytosolic form of LDH. To this end, an RNAi construct specific for cytosolic LDH is delivered to 832/13 cells and normal rat islets. The effects on cytosolic LDH activity and glucose stimulated insulin secretion are assessed.

The foregoing is illustrative of the present invention, and is not to be construed as limiting thereof. The invention is defined by the following claims, with equivalents of the claims to be included therein.

1. A method of enhancing fuel-stimulated insulin secretion in a subject, comprising administering to the subject a compound that modulates lactate dehydrogenase (LDH) activity in an amount effective to modulate LDH activity, thereby enhancing fuel-stimulated insulin secretion.

2. The method of claim 1, wherein the compound modulates LDH activity in the cytoplasm.

3. The method of claim 1, wherein the compound modulates LDH activity within the mitochondria.

4. The method of claim 1, wherein the method comprises a method of enhancing glucose-stimulated insulin secretion.

5. The method of claim 1, wherein the compound enhances LDH activity.

6. The method of claim 5, wherein the compound is an LDH activator.

7. The method of claim 1, wherein the compound is a polypeptide.

8. The method of claim 7, wherein the compound is an antibody that binds to LDH.

9. The method of claim 1, wherein the compound is a nucleic acid molecule.

10. The method of claim 9, wherein the compound is a DNA molecule.

11. The method of claim 9, wherein the compound is an RNA molecule.

12. The method of claim 11, wherein the compound is selected from the group consisting of an antisense RNA, an inhibitory RNA (RNAi) and a ribozyme.

13. The method of claim 1, wherein the subject is a human subject.

14. The method of claim 1, wherein the subject has impaired glucose tolerance.

15. The method of claim 14, wherein the subject has been diagnosed with diabetes mellitus.

16. The method of claim 14, wherein the subject is obese.

17. The method of claim 1, wherein the compound is identified by a process comprising:
contacting a LDH polypeptide with a test compound under conditions whereby modulation of the activity of the LDH polypeptide can be detected; and
detecting modulation of the activity of the LDH polypeptide, thereby identifying a compound that can modulate fuel-stimulated insulin secretion.

18. The method of claim 1, wherein the compound is identified by a process comprising:
introducing a test compound into a cell that comprises LDH polypeptide under conditions whereby modulation of the activity of the LDH polypeptide can be detected; and
detecting modulation of the activity of the LDH polypeptide, thereby identifying a compound that can modulate fuel-stimulated insulin secretion.

19. The method of claim 18, wherein the cell further comprises an isolated nucleic acid comprising a nucleotide sequence encoding LDH polypeptide and wherein the nucleotide sequence is expressed to produce LDH polypeptide.

20. The method of claim 1, wherein the compound is identified by a process comprising:
introducing a test compound into a cell that is capable of producing and secreting insulin and which comprises LDH polypeptide under conditions whereby modulation of fuel-stimulated insulin secretion can be detected; and
detecting modulation of fuel-stimulated insulin secretion, thereby identifying a compound that can modulate fuel-stimulated insulin secretion.

21. The method of claim 20, wherein the cell further comprises an isolated nucleic acid comprising a nucleotide sequence encoding LDH polypeptide and wherein the nucleotide sequence is expressed to produce LDH polypeptide.

22. The method of claim 1, wherein the compound is identified by a process comprising:
administering a test compound to a transgenic non-human mammal comprising an isolated nucleic acid encoding LDH polypeptide operably associated with a transcriptional control element functional in pancreatic islet β-cells, wherein the isolated nucleic acid is stably incorporated into and expressed in pancreatic islet β-cells of the transgenic non-human mammal under conditions whereby enhancement of fuel-stimulated insulin secretion can be detected; and
detecting enhancement of fuel-stimulated insulin secretion in the transgenic non-human mammal, thereby identifying a compound that can enhance fuel-stimulated insulin secretion.

23. A method of identifying a compound that can modulate fuel-stimulated insulin secretion, comprising:
contacting a lactate dehydrogenase (LDH) polypeptide with a test compound under conditions whereby modulation of the activity of the LDH polypeptide can be detected; and
detecting modulation of the activity of the LDH polypeptide, thereby identifying a compound that can modulate fuel-stimulated insulin secretion.

24. The method of claim 23, wherein the method comprises a method of identifying a compound that can modulate glucose-stimulated insulin secretion.

25. The method of claim 23, wherein the test compound enhances the activity of the LDH polypeptide.

26. The method of claim 23, wherein the test compound inhibits the activity of the LDH polypeptide.

27. The method of claim 23, wherein the LDH polypeptide is a cytoplasmic LDH.

28. The method of claim 23, wherein the LDH polypeptide is a mitochondrial LDH.

29. A method of identifying a compound that can modulate fuel-stimulated insulin secretion, comprising:
introducing a test compound into a cell that comprises lactate dehydrogenase (LDH) polypeptide under conditions whereby modulation of the activity of the LDH polypeptide can be detected; and
detecting modulation of the activity of the LDH polypeptide, thereby identifying a compound that can modulate fuel-stimulated insulin secretion.

30. The method of claim 29, wherein the cell further comprises an isolated nucleic acid comprising a nucleotide sequence encoding LDH polypeptide and wherein the nucleotide sequence is expressed to produce LDH polypeptide.

31. The method of claim 30, wherein the cell is stably transformed with the isolated nucleic acid.

32. The method of claim 30, wherein the isolated nucleic acid comprises a nucleotide sequence selected from the group consisting of:
(a) a nucleotide sequence selected from the group consisting of the nucleotide sequence of SEQ ID NO:1, SEQ ID NO:3, SEQ ID NO:24 and SEQ ID NO:26;
(b) a nucleotide sequence that hybridizes to a nucleotide sequence selected from the group consisting of the nucleotide sequence of SEQ ID NO:1, SEQ ID NO:3, SEQ ID NO:24 and SEQ ID NO:26 or its complementary nucleotide sequence under stringent conditions, wherein said nucleotide sequence encodes a functional LDH_{A} polypeptide; and
(c) a nucleotide sequence encoding an amino acid sequence encoded by the nucleotide sequences of (a) or (b), but which has a different nucleotide sequence than the nucleotide sequences of (a) or (b) due to the degeneracy of the genetic code or the presence of non-translated nucleotide sequences.

33. The method of claim 32, wherein the nucleotide sequence encodes an amino acid sequence having at least about 70% amino acid sequence similarity to an amino acid sequence selected from the group consisting of the amino acid sequence of SEQ ID NO:4, SEQ ID NO:25 and SEQ ID NO:27 or a functional fragment of any of the foregoing.

34. The method of claim 32, wherein the LDH polypeptide comprises an amino acid sequence selected from the group consisting of the amino acid sequence of SEQ ID NO:4, SEQ ID NO:25 and SEQ ID NO:27 or a functional fragment of any of the foregoing.

35. The method of claim 34, wherein the nucleotide sequence comprises a cDNA having the nucleotide sequence of SEQ ID NO:3.

36. The method of claim 32, wherein the cell is selected from the group consisting of a pancreatic islet β-cell and an insulinoma cell.

37. A method of identifying a compound that can modulate fuel-stimulated insulin secretion, comprising:
introducing a test compound into a cell that is capable of producing and secreting insulin and which comprises lactate dehydrogenase (LDH) polypeptide under conditions whereby modulation of fuel-stimulated insulin secretion can be detected; and
detecting modulation of fuel-stimulated insulin secretion, thereby identifying a compound that can modulate fuel-stimulated insulin secretion.

38. A transgenic non-human mammal comprising an isolated nucleic acid encoding lactate dehydrogenase (LDH) polypeptide operably associated with a transcriptional control element functional in pancreatic islet β-cells, wherein said isolated nucleic acid is stably incorporated into and expressed in pancreatic islet β-cells of said transgenic non-human mammal.

39. The transgenic non-human mammal of claim 38, wherein the non-human mammal is a mouse.

40. The transgenic non-human mammal of claim 38, wherein the non-human mammal has impaired glucose tolerance.

41. The transgenic non-human mammal of claim 36, wherein the LDH polypeptide is a human LDH.

42. A method of identifying a compound that can enhance fuel-stimulated insulin secretion, comprising:
administering a test compound to the transgenic non-human mammal of claim 38 under conditions whereby enhancement of fuel-stimulated insulin secretion can be detected; and
detecting enhancement of fuel-stimulated insulin secretion in the transgenic non-human animal, thereby identifying a compound that can enhance fuel-stimulated insulin secretion.

43. A method of enhancing fuel-stimulated insulin secretion in a mammalian subject comprising, administering to the mammalian subject an isolated nucleic acid comprising a nucleotide sequence encoding lactate dehydrogenase (LDH) polypeptide in an amount effective to enhance fuel-stimulated insulin secretion.

44. The method of claim 43, wherein the method comprises a method of enhancing glucose-stimulated insulin secretion.

45. The method of claim 43, wherein the isolated nucleic acid encoding LDH polypeptide is delivered to pancreatic islet β-cells.

46. The method of claim 43, wherein the LDH polypeptide is a cytoplasmic LDH.

47. The method of claim 43, wherein the LDH polypeptide is a mitochondrial LDH.

48. The method of claim 43, wherein the isolated nucleic acid comprises a nucleotide sequence selected from the group consisting of:
(a) a nucleotide sequence selected from the group consisting of the nucleotide sequence of SEQ ID NO:1, SEQ ID NO:3, SEQ ID NO:24 and SEQ ID NO:26;
(b) a nucleotide sequence that hybridizes to a nucleotide sequence selected from the group consisting of the nucleotide sequence of SEQ ID NO:1, SEQ ID NO:3, SEQ ID NO:24 and SEQ ID NO:26 or its complementary nucleotide sequence under stringent conditions, wherein said sequence encodes a functional LDH_{A} polypeptide; and
(c) a nucleotide sequence encoding an amino acid sequence encoded by the nucleotide sequences of (a) or (b), but which has a different nucleotide sequence than the sequences of (a) or (b) due to the degeneracy of the genetic code or the presence of non-translated nucleotide sequences.

49. The method of claim 43, wherein the nucleic acid further comprises a transcriptional control element functional in pancreatic islet β-cells and which is operably associated with a nucleotide sequence encoding LDH polypeptide.

50. The method of claim 43, wherein the nucleic acid is administered to the subject in a delivery vector.

51. A method of treating non-insulin dependent diabetes mellitus, comprising administering to a subject afflicted with non-insulin dependent diabetes mellitus an isolated nucleic acid comprising a nucleotide sequence encoding lactate dehydrogenase (LDH) polypeptide in a therapeutically effective amount.

52. The method of claim 51, wherein the subject is a human subject.

53. An isolated nucleic acid comprising a nucleotide sequence encoding a mitochondrial lactate dehydrogenase-A (LDH_{A}) polypeptide.

54. The isolated nucleic acid of claim 53, wherein said isolated nucleic acid comprises a nucleotide sequence selected from the group consisting of:
(a) a nucleotide sequence selected from the group consisting of the nucleotide sequence of SEQ ID NO:3, SEQ ID NO:24 and SEQ ID NO:26;
(b) a nucleotide sequence that hybridizes to a nucleotide sequence selected from the group consisting of the nucleotide sequence of SEQ ID NO:3, SEQ ID NO:24 and SEQ ID NO:26 or its complementary nucleotide sequence under stringent conditions, wherein said nucleotide sequence encodes a functional mitochondrial LDH_{A} polypeptide; and
(c) a nucleotide sequence encoding an amino acid sequence encoded by the nucleotide sequences of (a) or (b), but which has a different nucleotide sequence than the nucleotide sequences of (a) or (b) due to the degeneracy of the genetic code or the presence of non-translated nucleotide sequences.

55. The isolated nucleic acid of claim 53, wherein said nucleotide sequence encodes an amino acid sequence having at least about 70% amino acid sequence similarity to an amino acid sequence selected from the group consisting of SEQ ID NO:4, SEQ ID NO:25 and SEQ ID NO:27 or a functional fragment of any of the foregoing.

56. The isolated nucleic acid of claim 53, wherein said nucleotide sequence encodes an amino acid sequence comprising an amino acid sequence selected from the group consisting of SEQ ID NO:4, SEQ ID NO:25 and SEQ ID NO:27 or a functional fragment of any of the foregoing.

57. A vector comprising the isolated nucleic acid of claim 53.

58. An isolated nucleic acid comprising a nucleotide sequence consisting essentially of a nucleotide sequence selected from the group consisting of the nucleotide sequence of SEQ ID NO:3, SEQ ID NO:24 and SEQ ID NO:26.

59. An isolated mitochondrial lactate dehydrogenase-A (LDH_{A}) polypeptide.

60. A tetrameric LDH comprising the LDH_{A} polypeptide of claim 59.

61. The isolated LDH_{A} polypeptide of claim 59, wherein said polypeptide comprises an amino acid sequence having at least about 70% amino acid sequence similarity to an amino acid sequence selected from the group consisting of SEQ ID NO:4, SEQ ID NO:25 and SEQ ID NO:27 or a functional fragment of any of the foregoing.

62. The isolated LDH_{A} polypeptide of claim 59, wherein said polypeptide comprises an amino acid sequence selected from the group consisting of SEQ ID NO:4, SEQ ID NO:25 and SEQ ID NO:27 or a functional fragment thereof.

63. An isolated LDH_{A} polypeptide consisting essentially of an amino acid sequence selected from the group consisting of SEQ ID NO:4, SEQ ID NO:25 and SEQ ID NO:27 or a functional fragment thereof.

64. A cultured cell for use in a cell-based screening assay comprising the isolated nucleic acid of claim 53.

65. A cultured cell for use in a cell-based screening assay comprising the isolated nucleic acid of claim 58.

66. A cultured cell for use in a cell-based screening assay comprising the isolated mitochondrial LDH_{A} polypeptide of claim 59.

67. A cultured cell for use in a cell-based screening assay comprising the isolated mitochondrial LDH_{A} polypeptide of claim 63.
